(19) United States
(12) Patent Application Publication
(10) Pub. No.: US 2015/0152499 A1
(43) Pub. Date: Jun. 4, 2015

(54) DIAGNOSTIC PORTFOLIO AND ITS USES

(71) Applicant: INTERNA TECHNOLOGIES B.V., Nijmegen (NL)

(72) Inventors: Anton Jan Van Zonneveld, Soest (NL); Jacques Duijs, Oegstgeest (NL); Johan Kuiper, Gouda (NL); Thijs Jan Zweers, Leiden (NL); Johan Wouter Jukema, Oegstgeest (NL); Eugene Berezikov, Bedum (NL)

(73) Assignee: InteRNA Technologies B.V., Nijmegen (NE)

(21) Appl. No.: 14/409,947

(22) PCT Filed: Jul. 3, 2013
(86) PCT No.: PCT/NL2013/050491

§ 371 Date: Dec. 19, 2014

Related U.S. Application Data
(60) Provisional application No. 61/667,524, filed on Jul. 3, 2012.

(30) Foreign Application Priority Data
Jul. 3, 2012 (EP) 12174814.9

(51) Int. Cl. C12Q 1/68

(52) U.S. Cl. CPC C12Q 1/6883; C12Q 2600/118; C12Q 2600/178; C12Q 2600/158

(57) ABSTRACT

A diagnostic portfolio comprising or consisting of isolated nucleic acid molecules, their complement and/or fragments thereof, said nucleic acid molecules, complements, equivalents and/or fragments thereof being represented by sequences comprising or consisting of sequences having at least 80% of sequence identity with SEQ ID NO:1-92, or 49-140 or derived thereof (i.e. groups a), b), c), d), e), f), g), h), i) or j) or subgroups thereof) and their uses.

### FIELD OF THE INVENTION

The invention relates to a diagnostic portfolio comprising or consisting of isolated nucleic acid molecules, complements, equivalents and/or fragments thereof, said nucleic acid molecules being represented by sequences comprising or consisting of sequences having at least 80% of sequence identity with sequences selected from the groups consisting of SEQ ID NO:1-92, or of 49-140 or derived thereof (i.e. groups a), b), c), d), e), f), g), h), i) or j) or subgroups thereof) and their uses.

### BACKGROUND OF THE INVENTION

Atherosclerosis and ischemic vascular disease is not only the result of defects in the vascular wall but also highly dependent on the altered presence or dysfunction of various subsets of circulating bone-marrow derived CD14⁺ myeloid and CD4⁺ and CD8⁺ lymphocytes cells that normally function in vascular maintenance and repair. These changes in the phenotype composition of these circulating cells are determined by local and systemic factors associated with risk factors for cardiovascular disease (FIG. 1). As the human population has a high level of genetic variation, the hematopoietic system of different individuals will respond to a highly variable degree on the pro-inflammatory drivers associated with cardiovascular risk factors. Therefore, phenotypic alterations in circulating CD14⁺ myeloid and CD4⁺ and CD8⁺ lymphocytes directly reflect the susceptibility of an individual to the development of cardiovascular disease or other diseases in which these cell types are involved such as fibrosis or cancer.

MicroRNAs (miRNAs) are highly conserved, comprises approximately 22 nucleotide non-coding RNAs regulators of gene expression that play a major role in hematopoietic lineage development (Havelange V et al, 2010) MiRNAs coordinate coherent signal transduction pathways by regulating multiple genes within a single cell type (Havelange V et al, 2010 and Carthew R W et al, 2009) Also miRNA have been demonstrated to facilitate many aspects of the cellular inflammatory responses (O'Neill L A et al, 2011).

Hence, miRNA expression profiles of circulating CD14⁺ myeloid and CD4⁺ and CD8⁺ lymphocytes will directly reflect the differentiation state or phenotype of these cells. In this way, miRNA signatures relate to the impact a particular cardiovascular risk factor has on these cells and thus on the susceptibility of the particular individual to cardiovascular disease. As the expression of miRNAs may be dependent on genetic polymorphisms present in the donors, as many miRNAS can be expressed in a particular cell and not all miRNAs will be related to the phenotypic changes that relate to increased risk for disease, we have designed a strategy to specifically select phenotype-specific miRNAs from the circulating hematopoietic cells that play a key role in directing artherogenesis and neovascularization. These phenotype-selective miRNA sets offer an optimal platform for the identification of individuals at risk for the development of cardiovascular disease. We demonstrate that when miRNA profiles are generated to identify patients at risk for the development of cardiovascular disease the combination of the phenotype-selective miRNAs give better prediction then individual or low numbers of miRNAs.

### DESCRIPTION OF THE INVENTION

### Diagnostic Portfolio

A first aspect of the invention relates to a diagnostic portfolio comprising or consisting of nucleic acid molecules, complements, equivalents, and/or fragments thereof, said nucleic acid molecules, complements, equivalents and/or fragments thereof being represented by sequences comprising or consisting of sequences having at least 80% of sequence identity with sequences selected from the groups consisting of SEQ ID NO:1-92, or of 49-140 or subgroups thereof defined later herein (i.e. a), b), c), d), e), f), g), h) i) or j)).

Complement, equivalent, fragment are all later defined herein.

Within the context of the invention, a diagnostic portfolio may comprise a combination of nucleic acid molecules, complements, equivalents and/or fragments thereof selected from the groups consisting of nucleic acid molecules being represented by sequences comprising or consisting of sequences having at least 80% of sequence identity with SEQ ID NO:1-92, or of 49-140 or subgroups thereof as defined later herein (i.e. a), b), c), d), e), f), g), h), i) or j)).

Each combination of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 till 92 nucleic acid molecules, complements and/or fragments thereof for the first group (i.e. SEQ ID NO:1-92) and/or of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 till 92 nucleic acid molecules, complements and/or fragments thereof for the second group (i.e. SEQ ID NO:49-140) may be used. In a preferred embodiment, the 92 nucleic acid molecules, complements, equivalents and/or fragments thereof of the first group are being used. In another preferred embodiment, the 92 nucleic acid molecules, complements, equivalents and/or fragments thereof of the second group are being used. In another preferred embodiment, all the nucleic acid molecules, complements, equivalents and/or fragments thereof of both groups (140 in total) are used. Table 3 identifies preferred SEQ ID NO of all nucleic acid of the first group. Table 4 identifies preferred SEQ ID NO of all nucleic acid of the second group. Both groups of nucleic acids are predictive for inflammation in a subject.

The first group (SEQ ID NO:1-92) has been identified based on nucleic acid molecules whose expression is modulated or differentially expressed in circulating CD14⁺ cells or monocytes characterized as intermediate or non-classical monocytes and that are predictive of inflammation (example 1). The three major subsets of circulating CD14⁺ cells are defined as classical (CD14⁺⁺CD16⁻) monocytes, intermediate monocytes (CD14⁺⁺CD16⁺) and non-classical (CD14⁺CD16⁺⁺) monocytes and define the global CD14⁺ population having a global miRNA expression profile. Intermediate and non-classical monocytes define a group of monocytes that are predictive for inflammation in the context of the invention.

The second group (SEQ ID NO:49-140) has been identified based on nucleic acid molecules whose expression is modulated or differentially expressed in circulating CD4⁺ and CD8⁺ cells and reflect the activation state of components of the acquired immune system. (example 2).

A preferred diagnostic portfolio of the invention comprises more than one, more preferably at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 till 92 nucleic acid molecules, complements, equivalents and/or fragments thereof as defined in the first diagnostic portfolio (i.e. sequences comprising or consisting of sequences having at least 80% of sequence identity with sequences selected from the group consisting of SEQ ID NO: 1-92).

A more preferred diagnostic portfolio is derived from the first diagnostic portfolio as explained below. This more preferred diagnostic portfolio is selected from the following groups:

a) at least one isolated nucleic acid molecule, complement, equivalent and/or fragment thereof, said nucleic acid molecule, complement, equivalent and/or fragment thereof being selected from the group consisting of: miR-449a, miR-212, miR-132, miR-342-3p, mir-146a and mir-590-5p (i.e. selected from sequences comprising or consisting of sequences having at least 80% of sequence identity with sequences selected from the group consisting of SEQ ID NO: 29, 33, 43, 68, 71 and 90 see table 5),
b) at least one isolated nucleic acid molecule, complement, equivalent and/or fragment thereof, said nucleic acid molecule, complement, equivalent and/or fragment thereof being selected from the group consisting of: miR-449b, miR-487b, miR-200a, miR-210, miR-708 and miR-376c (i.e. selected from sequences comprising or consisting of sequences having at least 80% of sequence identity with sequences selected from the group consisting of SEQ ID NO: 27, 34, 37, 45, 47, and 79, see table 6),
c) at least one, preferably at least two isolated nucleic acid molecule, complement, equivalent and/or fragment thereof, said nucleic acid molecule, complement, equivalent and/or fragment thereof being selected from the group consisting of: miR-133a, miR-10a, miR-34a, miR-32, miR191, miR885-5p, miR125a-5p, miR-99b, miR-146b-5p, miR-130a, miR-100, miR-130b, miR-486-3p, miR-500, miR-128, miR-145, miR-221, miR-574-3p, miR-19a, miR-19b, miR-365, miR-345, miR-20a, miR-93, miR-20b, miR-223, miR-17, miR193a-5p, miR-374b and miR-628-5p (i.e. selected from sequences comprising or consisting of sequences having at least 80% of sequence identity with sequences selected from the group consisting of: SEQ ID NO: 3, 10, 14, 20, 46, 62, 66, 72, 75 15, 17, 18, 19, 25, 26, 30, 39, 42, 44, 52, 53, 54, 55, 56, 63, 80, 81, 86, 88 and 91 see table 7),
d) at least one isolated nucleic acid molecule, complement, equivalent and/or fragment thereof, said nucleic acid molecule, complement, equivalent and/or fragment thereof being selected from the group consisting of hsa-miR-let-7d, hsa-miR-let-7e, hsa-miR-19a, hsa-miR-145, hsa-miR-191, hsa-miR-193a-5p, hsa-miR-195, hsa-miR-197, hsa-miR-221, hsa-miR-223, hsa-miR-365, hsa-miR-422a, hsa-miR-501-5p, hsa-miR-574-3p, hsa-miR-628-5p, hsa-miR-15b and hsa-miR-130a (i.e. selected from sequences comprising or consisting of sequences having at least 80% of sequence identity with sequences selected from the group consisting of: SEQ ID NO: 49, 50, 53, 25, 75, 26, 77, 78, 80, 81, 30, 31, 40, 42, 44, 4 and 18 (table 8)),
e) at least one isolated nucleic acid molecule, complement, equivalent and/or fragment thereof, said nucleic acid molecule, complement, equivalent and/or fragment thereof being selected from the group consisting of hsa-miR-let-7e, hsa-miR-let-7f, hsa-miR-20a, hsa-miR-28-5p, hsa-miR-124, hsa-miR-126, hsa-miR-128, hsa-miR-132, hsa-miR-133b, hsa-miR-150, hsa-miR-191, hsa-miR-223, hsa-miR-342-3p, hsa-miR-365, hsa-miR-424, hsa-miR-218 and hsa-miR-449a (i.e. selected from sequences comprising or consisting of sequences having at least 80% of sequence identity with sequences selected from the group consisting of: SEQ ID NO: 50, 2, 55, 9, 48, 67, 17, 68, 21, 73, 75, 81, 29, 30, 32, 28 and 33 (table 8)).
f) at least one isolated nucleic acid molecule, complement, equivalent and/or fragment thereof, said nucleic acid molecule, complement, equivalent and/or fragment thereof being selected from the group consisting of hsa-miR-18b, hsa-miR-146-5p, hsa-miR-422a, hsa-miR-424, has-miR-138 and has-miR-218 (i.e. selected from sequences comprising or consisting of sequences having at least 80% of sequence identity with sequences selected from the group consisting of: SEQ ID NO: 5, 72, 31, 32, 22 and 28 (table 8)),
g) at least one isolated nucleic acid molecule, complement, equivalent and/or fragment thereof, said nucleic acid molecule, complement, equivalent and/or fragment thereof being selected from the group consisting of hsa-miR-31, hsa-miR-99a, hsa-miR-126, hsa-miR-150, hsa-miR-125a-5p, hsa-miR-19b, hsa-miR-133b, and hsa-miR-223 (i.e. selected from sequences comprising or consisting of sequences having at least 80% of sequence identity with sequences selected from the group consisting of: SEQ ID NO: 61, 13, 67, 73, 66, 54, 21 and 81 (table 8)),
h) at least one isolated nucleic acid molecule, complement, equivalent and/or fragment thereof, said nucleic acid molecule, complement, equivalent and/or fragment thereof being selected from the group consisting of hsa-miR-31, hsa-miR-99a, hsa-miR-126, and hsa-miR-150 (i.e. selected from sequences comprising or consisting of sequences having at least 80% of sequence identity with sequences selected from the group consisting of: SEQ ID NO: 61, 13, 67, and 73 (table 8)),
i) at least one isolated nucleic acid molecule, complement, equivalent and/or fragment thereof, said nucleic acid molecule, complement, equivalent and/or fragment thereof being selected from the group consisting of hsa-miR-133b, hsa-miR-486-3p and hsa-miR-671-3p (i.e. selected from sequences comprising or consisting of sequences having at least 80% of sequence identity with sequences selected from the group consisting of: SEQ ID NO: 21, 88 and 89 (table 8)),
j) at least one isolated nucleic acid molecule, complement, equivalent and/or fragment thereof, said nucleic acid molecule, complement, equivalent and/or fragment thereof being selected from the group consisting of hsa-miR-376c and has-miR-885-5p (i.e. selected from sequences comprising or consisting of sequences having at least 80% of sequence identity with sequences selected from the group consisting of: SEQ ID NO: 47 and 46 (table 8).

The group identified in a) above, comprises nucleic acid molecules that were modulated, or differentially expressed, preferably up regulated in intermediate monocytes (CD14⁺⁺CD16⁺) and non-classical (CD14⁺CD16⁺⁺) monocytes compared to expression in classical monocytes (CD14⁺⁺CD16⁻) and/or in global CD14⁺ miRNA expression profiles. Each nucleic acid molecule from this group is therefore assumed to be linked or associated with the presence or absence of inflammation.

The group identified in b) above, comprises nucleic acid molecules that were modulated, or differentially expressed in intermediate monocytes (CD14⁺⁺CD16⁺) and non-classical (CD14⁺CD16⁺⁺) monocytes compared to expression in classical monocytes (CD14⁺⁺CD16⁻) and/or in global CD14⁺ miRNA expression profiles. Each nucleic acid molecule from this group is therefore assumed to linked or associated with the presence or absence of inflammation.

The group identified in c) above, comprises nucleic acid molecules that were modulated, or differentially expressed, preferably down regulated in intermediate monocytes (CD14⁺⁺CD16⁺) and non-classical (CD14⁺CD16⁺⁺) monocytes compared to expression in classical monocytes (CD14⁺⁺CD16⁻) and/or in global CD14⁺ miRNA expression profiles. Each nucleic acid molecule from this group is therefore assumed to linked or associated with the presence or absence of inflammation.

The group identified in d) above, comprises nucleic acid molecules that were modulated, or differentially expressed, preferably up regulated in subjects suspected to develop or having a vessel disease. A vessel disease is a vascular disease that may affect any type of blood vessel including artery. A vascular disease is a form of a cardiovascular disease. A vessel disease may also include stenosis in main arteries such as the kidney artery or the carotids or may affect peripheral arteries. Critical limb ischemia is also encompassed within the scope of vessel disease. Each nucleic acid molecule from this group is therefore assumed to be linked or associated with the presence or absence of inflammation in the context of such a vessel disease. Currently, a vessel disease may be diagnosed using angiography (quantified using the SYNTAX score, Sjanos G et al). The diagnostic method of the invention is quite attractive since it is less invasive and less expensive than a diagnostic method based on the use of angiography methods. The use of group d) of the invention allows an early, cheap, easy and specific diagnostic of such kind of disease or condition.

The group identified in e) above, comprises nucleic acid molecules that were modulated, or differentially expressed, preferably up regulated in subjects suspected to having a stable CAD (Coronary Artery Disease). CAD also named atherosclerotic heart disease or angina pectoris is the most common type of heart disease and cause of heart attacks. The disease is caused by plaque building up along the inner walls of the arteries of the heart, which narrows the arteries and restricts blood flow to the heart. It is the leading cause of death worldwide. In a stable CAD, subjects only experienced chest pain and associated symptoms during activity. Unstable CAD, is a more severe form of CAD than stable CAD since it manifest itself at rest and can be progressive. Each nucleic acid molecule from this group is therefore assumed to be linked or associated with the presence or absence of inflammation in the context of such a stable CAD. CAD is currently diagnosed using electrocardiography (ECG), coronary angiography, intravascular ultrasound or magnetic resonance imaging (MRI). The diagnostic method of the invention is quite attractive since it is less invasive and less expensive than a diagnostic method based on the use of angiography methods. The use of group e) of the invention allows an early, cheap, easy and specific diagnostic of such kind of disease or condition. In addition, it is quite attractive to be able to distinguish subjects suspected to have a stable CAD versus unstable CAD in view of the differences of the two conditions explained above.

The group identified in f) above, comprises nucleic acid molecules that were modulated, or differentially expressed, preferably up regulated in subjects suspected to have systemic inflammation possibly with modulated or differentially expressed, preferably upregulated CRP (also named CRP group). C-reactive protein (CRP) is a sensitive but non-specific marker for inflammation. Elevated CRP blood levels, especially measured with high-sensitivity assays, can predict the risk of MI, as well as stroke and development of diabetes.

Each nucleic acid molecule from this group is therefore assumed to be linked or associated with the presence or absence of systemic inflammation and may correlate with the expression of CRP or elevated expression thereof. While CRP may be directly assessed as a marker of systemic inflammation, it is not believed to be specific for cardiovascular complications that may result from systemic inflammation. It is therefore anticipated that a diagnostic method of the invention based on the use of the group f) is more specific for the assessment of systemic inflammation, and for the assessment of cardiovascular complications that may result from such systemic inflammation than a diagnostic method based on the assessment of CRP.

The group identified in g) above, comprises nucleic acid molecules that were modulated, or differentially expressed, preferably up regulated in subjects suspected to develop or having diabetes. Diabetes is a condition or disease associated with elevated or abnormally elevated levels of blood glucose such as associated with metabolic syndrome and diabetes mellitus type-2. Each nucleic acid molecule from this group is therefore assumed to be linked or associated with the presence or absence of inflammation in the context of such disease. The use of group g) of the invention allows an early and specific diagnostic of such kind of disease or condition.

The group identified in h) above, comprises nucleic acid molecules that were modulated, or differentially expressed, preferably up regulated in female subjects suspected to develop or having diabetes. Each nucleic acid molecule from this group is therefore assumed to be linked or associated with the presence or absence of inflammation in the context of such a disease in females. The use of group h) of the invention allows an early and specific diagnostic of such kind of disease or condition.

The group identified in i) above, comprises nucleic acid molecules that were modulated, or differentially expressed, preferably up regulated in subjects suspected to develop or having familial hypercholesterolemia. Familial hypercholesterolemia is a genetic disorder associated with high or abnormal high levels of circulating LDL cholesterol. Each nucleic acid molecule from this group is therefore assumed to be linked or associated with the presence or absence of inflammation in the context of such a disease. The use of group i) of the invention allows an early and specific diagnostic of such kind of disease or condition.

The group identified in j) above, comprises nucleic acid molecules that were modulated, or differentially expressed, preferably up regulated in subjects suspected to develop or having an increased number of diseased coronary vessels. A method based on the use of this group is expected to have the same advantages as a method based on the use of groups d) or e).

An even more preferred diagnostic portfolio is derived from the following groups:

a) at least one, 2, 3, 4, 5 or 6 nucleic acid molecule, complement, equivalent and/or fragment thereof, said nucleic acid molecule, complement, equivalent and/or fragment thereof being selected from the group consisting of: miR-449a, miR-212, miR-132, miR-342-3p, mir-146a and mir-590-5p, (i.e. selected from sequences comprising or consisting of sequences having at least 80% of sequence identity with sequences selected from the group consisting of: SEQ ID NO: 29, 33, 43, 68, 71 and 90),
b) at least one, 2, 3, 4, 5 or 6 isolated nucleic acid molecule, complement, equivalent and/or fragment thereof, said nucleic acid molecule, complement, equivalent and/or fragment thereof being selected from the group consisting of: miR-449b, miR-487b, miR200a, miR-210, miR708 and miR-376c (i.e. selected from sequences comprising or consisting of sequences having at least 80% of sequence identity with sequences selected from the group consisting of SEQ ID NO: 27, 34, 37, 45, 47, and 79),
c) at least one, 2, 3, 4, 5, 6, 7, 8, 9, 10, 11, 12, 13, 14, 15, 16, 17, 18, 19, 20, 21, 22, 23, 24, 25, 26, 27, 28, 29, 30 or 30 isolated nucleic acid molecule, complement, equivalent and/or fragment thereof, said nucleic acid molecule, complement, equivalent and/or fragment thereof being selected from the group consisting of: miR-133a, miR-10a, miR-34a, miR-32, miR191, miR885-5p, miR125a-5p, miR99b, miR146b-5p, miR-130a, miR-100, miR-130b, miR-486-3p, miR-500, miR128, miR-145, miR-221, miR-5′74-3p, miR-19a, miR-19b, miR-365, miR-345, miR-20a, miR-93, miR-20b, miR-223, miR-17, miR193a-5p, miR374b and miR-628-5p (i.e. selected from sequences comprising or consisting of sequences having at least 80% of sequence identity with sequences selected from the group consisting of: SEQ ID NO: 3, 10, 14, 20, 46, 62, 66, 72, 75, 15, 17, 18, 19, 25, 26, 30, 39, 42, 44, 52, 53, 54, 55, 56, 63, 80, 81, 86, 88 and 91).
d) at least one, 2, 3, 4, 5, 6, 7, 8, 9, 10, 11, 12, 13, 14, 15, 16, 17 isolated nucleic acid molecule, complement, equivalent and/or fragment thereof, said nucleic acid molecule, complement, equivalent and/or fragment thereof being selected from the group consisting of hsa-miR-let-7d, hsa-miR-let-7e, hsa-miR-19a, hsa-miR-145, hsa-miR-191, hsa-miR-193a-5p, hsa-miR-195, hsa-miR-197, hsa-miR-221, hsa-miR-223, hsa-miR-365, hsa-miR-422a, hsa-miR-501-5p, hsa-miR-574-3p, hsa-miR-628-5p, hsa-miR-15b and hsa-miR-130a (i.e. selected from sequences comprising or consisting of sequences having at least 80% of sequence identity with sequences selected from the group consisting of: SEQ ID NO: 49, 50, 53, 25, 75, 26, 77, 78, 80, 81, 30, 31, 40, 42, 44, 4 and 18 (table 8)),
e) at least one, 2, 3, 4, 5, 6, 7, 8, 9, 10, 11, 12, 13, 14, 15, 16, 17 isolated nucleic acid molecule, complement, equivalent and/or fragment thereof, said nucleic acid molecule, complement, equivalent and/or fragment thereof being selected from the group consisting of hsa-miR-let-7e, hsa-miR-let-7f hsa-miR-20a, hsa-miR-28-5p, hsa-miR-124, hsa-miR-126, hsa-miR-128, hsa-miR-132, hsa-miR-133b, hsa-miR-150, hsa-miR-191, hsa-miR-223, hsa-miR-342-3p, hsa-miR-365, hsa-miR-424, hsa-miR-218 and hsa-miR-449a (i.e. selected from sequences comprising or consisting of sequences having at least 80% of sequence identity with sequences selected from the group consisting of: SEQ ID NO: 50, 2, 55, 9, 48, 67, 17, 68, 21, 73, 75, 81, 29, 30, 32, 28 and 33 (table 8)).
f) at least one, 2, 3, 4, 5, 6 isolated nucleic acid molecule, complement, equivalent and/or fragment thereof, said nucleic acid molecule, complement, equivalent and/or fragment thereof being selected from the group consisting of hsa-miR-18b, hsa-miR-146-5p, hsa-miR-422a, hsa-miR-424, has-miR-138 and has-miR-218 (i.e. selected from sequences comprising or consisting of sequences having at least 80% of sequence identity with sequences selected from the group consisting of: SEQ ID NO: 5, 72, 31, 32, 22 and 28 (table 8)),
g) at least one at least one, 2, 3, 4, 5, 6, 7, 8, isolated nucleic acid molecule, complement, equivalent and/or fragment thereof, said nucleic acid molecule, complement, equivalent and/or fragment thereof being selected from the group consisting of hsa-miR-31, hsa-miR-99a, hsa-miR-126, hsa-miR-150, hsa-miR-125a-5p, hsa-miR-19b, hsa-miR-133b, hsa-miR-223 (i.e. selected from sequences comprising or consisting of sequences having at least 80% of sequence identity with sequences selected from the group consisting of: SEQ ID NO: 61, 13, 67, 73, 66, 54, 21 and 81 (table 8)),
h) at least one at least one, 2, 3, 4, isolated nucleic acid molecule, complement, equivalent and/or fragment thereof, said nucleic acid molecule, complement, equivalent and/or fragment thereof being selected from the group consisting of hsa-miR-31, hsa-miR-99a, hsa-miR-126, hsa-miR-150 (i.e. selected from sequences comprising or consisting of sequences having at least 80% of sequence identity with sequences selected from the group consisting of: SEQ ID NO: 61, 13, 67 and 73 (table 8)),
i) at least one, 2, 3 isolated nucleic acid molecule, complement, equivalent and/or fragment thereof, said nucleic acid molecule, complement, equivalent and/or fragment thereof being selected from the group consisting of hsa-miR-133b, hsa-miR-486-3p and hsa-miR-671-3p (i.e. selected from sequences comprising or consisting of sequences having at least 80% of sequence identity with sequences selected from the group consisting of: SEQ ID NO: 21, 88 and 89 (table 8)).
j) at least one or two isolated nucleic acid molecule, complement, equivalent and/or fragment thereof, said nucleic acid molecule, complement, equivalent and/or fragment thereof being hsa-miR-376c and/or has-miR-885-5p (i.e. selected from sequences comprising or consisting of sequences having at least 80% of sequence identity with sequences selected from the group consisting of: SEQ ID NO: 47 and 46 (table 8)).

An even more preferred subgroup in group e) comprises isolated nucleic acid molecule hsa-miR-132 and hsa-miR-342-3p, complement, equivalent and/or fragment thereof, (i.e. selected from sequences comprising or consisting of sequences having at least 80% of sequence identity with sequences SEQ ID NO: 68 and 29 respectively (table 8)). This subgroup is attractive since it gives a prediction as to status of CAD (i.e. stable versus unstable CAD). The advantage of such prediction has already been explained earlier herein.

An even more preferred subgroup in group g) comprises isolated nucleic acid molecule hsa-miR-223, complement, equivalent and/or fragment thereof (i.e. selected from sequences having at least 80% of sequence identity with sequences SEQ ID NO: 81 (table 8)). This subgroup is attractive since it also gives a prediction as to hypertension in females. Diabetes and hypertension are critical risk factors for diastolic heart failure with preservation of ejection fraction. This is the typical manifestation of heart disease in females. This is therefore quite attractive to have identified a single miR that is predictive for diabetes and hypertension in females.

An even more preferred diagnostic portfolio is derived from the following groups:

a) 6 nucleic acid molecules, complements, equivalents and/or fragments thereof, said nucleic acid molecules, complements, equivalents and/or fragments thereof being selected from the group consisting of: miR-449a, miR-212, miR-132, miR-342-3p, mir-146a and mir-590-5p (i.e. selected from sequences comprising or consisting of sequences having at least 80% of sequence identity with sequences selected from the group consisting of: SEQ ID NO: 29, 33, 43, 68, 71 and 90 see table 5),
b) 6 nucleic acid molecules, complements, equivalents and/or fragments thereof, said nucleic acid molecules, complements, equivalents and/or fragments thereof being selected from the group consisting of: miR-449b, miR-487b, miR200a, miR-210, miR708 and miR-376c (i.e. selected from sequences comprising or consisting of sequences having at least 80% of sequence identity with sequences selected from the group consisting of SEQ ID NO: 27, 34, 37, 45, 47, and 79),
c) 30 nucleic acid molecules, complements, equivalents and/or fragments thereof, said nucleic acid molecules, complements, equivalents and/or fragments thereof being selected from the group consisting of: miR-133a, miR-10a, miR-34a, miR-32, miR191, miR885-5p, miR125a-5p, miR99b, miR146b-5p, miR-130a, miR-100, miR-130b, miR-486-3p, miR-500, miR128, miR-145, miR-221, miR-574-3p, miR-19a, miR-19b, miR-365, miR-345, miR-20a, miR-93, miR-20b, miR-223, miR-17, miR193a-5p, miR374b and miR-628-5p (i.e. selected from sequences comprising or consisting of sequences having at least 80% of sequence identity with sequences selected from the group consisting of: SEQ ID NO: 3, 10, 14, 20, 46, 62, 66, 72, 75, 15, 17, 18, 19, 25, 26, 30, 39, 42, 44, 52, 53, 54, 55, 56, 63, 80, 81, 86, 88 and 91),
d) 17 isolated nucleic acid molecules, complements, equivalents and/or fragments thereof, said nucleic acid molecules, complements, equivalents and/or fragments thereof being selected from the group consisting of hsa-miR-let-7d, hsa-miR-let-7e, hsa-miR-19a, hsa-miR-145, hsa-miR-191, hsa-miR-193a-5p, hsa-miR-195, hsa-miR-197, hsa-miR-221, hsa-miR-223, hsa-miR-365, hsa-miR-422a, hsa-miR-501-5p, hsa-miR-5′74-3p, hsa-miR-628-5p, hsa-miR-15b and hsa-miR-130a (i.e. selected from sequences comprising or consisting of sequences having at least 80% of sequence identity with sequences selected from the group consisting of: SEQ ID NO: 49, 50, 53, 25, 75, 26, 77, 78, 80, 81, 30, 31, 40, 42, 44, 4 and 18 (table 8)),
e) 17 isolated nucleic acid molecules, complements, equivalents and/or fragments thereof, said nucleic acid molecules, complements, equivalents and/or fragments thereof being selected from the group consisting of hsa-miR-let-7e, hsa-miR-let-7f hsa-miR-20a, hsa-miR-28-5p, hsa-miR-124, hsa-miR-126, hsa-miR-128, hsa-miR-132, hsa-miR-133b, hsa-miR-150, hsa-miR-191, hsa-miR-223, hsa-miR-342-3p, hsa-miR-365, hsa-miR-424, hsa-miR-218 and hsa-miR-449a (i.e. selected from sequences comprising or consisting of sequences having at least 80% of sequence identity with sequences selected from the group consisting of: SEQ ID NO: 50, 2, 55, 9, 48, 67, 17, 68, 21, 73, 75, 81, 29, 30, 32, 28 and 33 (table 8)).
f) 6 isolated nucleic acid molecules, complements, equivalents and/or fragments thereof, said nucleic acid molecules, complements, equivalents and/or fragments thereof being selected from the group consisting of hsa-miR-18b, hsa-miR-146-5p, hsa-miR-422a, hsa-miR-424, has-miR-138 and has-miR-218 (i.e. selected from sequences comprising or consisting of sequences having at least 80% of sequence identity with sequences selected from the group consisting of: SEQ ID NO: 5, 72, 31, 32, 22 and 28 (table 8)),
g) 8 isolated nucleic acid molecules, complements, equivalents and/or fragments thereof, said nucleic acid molecules, complements, equivalents and/or fragments thereof being selected from the group consisting of hsa-miR-31, hsa-miR-99a, hsa-miR-126, hsa-miR-150, hsa-miR-125a-5p, hsa-miR-19b, hsa-miR-133b, hsa-miR-223 (i.e. selected from sequences comprising or consisting of sequences having at least 80% of sequence identity with sequences selected from the group consisting of: SEQ ID NO: 61, 13, 67, 73, 66, 54, 21 and 81 (table 8)),
h) 4 isolated nucleic acid molecules, complements, equivalents and/or fragments thereof, said nucleic acid molecules, complements, equivalents and/or fragments thereof being selected from the group consisting of hsa-miR-31, hsa-miR-99a, hsa-miR-126, hsa-miR-150 (i.e. selected from sequences comprising or consisting of sequences having at least 80% of sequence identity with sequences selected from the group consisting of: SEQ ID NO: 61, 13, 67 and 73 (table 8)),
i) 3 isolated nucleic acid molecules, complements, equivalents and/or fragments thereof, said nucleic acid molecules, complements, equivalents and/or fragments thereof being selected from the group consisting of hsa-miR-133b, hsa-miR-486-3p and hsa-miR-671-3p (i.e. selected from sequences comprising or consisting of sequences having at least 80% of sequence identity with sequences selected from the group consisting of: SEQ ID NO: 21, 88 and 89 (table 8)),
j) two isolated nucleic acid molecules, complements, equivalents and/or fragments thereof, said nucleic acid molecules, complements, equivalents and/or fragments thereof being selected from the group consisting of hsa-miR-376c and has-miR-885-5p (i.e. selected from sequences comprising or consisting of sequences having at least 80% of sequence identity with sequences selected from the group consisting of: SEQ ID NO: 47 and 46 (table 8)).

An even more preferred diagnostic portfolio is derived from the following groups:

a) 6 distinct nucleic acid molecules, complements, equivalents and/or fragments thereof, said nucleic acid molecules, complements, equivalents and/or fragments thereof being miR-449a, miR-212, miR-132, miR-342-3p, mir-146a and mir-590-5p (i.e. sequences having at least 80% of sequence identity with SEQ ID NO: 29, 33, 43, 68, 71 and 90 see table 5),
b) 6 distinct nucleic acid molecules, complements, equivalents and/or fragments thereof, said nucleic acid molecules, complements, equivalents and/or fragments thereof being miR-449b, miR-487b, miR200a, miR-210, miR708 and miR-376c (i.e. sequences having at least 80% of sequence identity with SEQ ID NO: 27, 34, 37, 45, 47, and 79),
c) 30 distinct nucleic acid molecules, complements, equivalents and/or fragments thereof, said nucleic acid molecules, complements, equivalents and/or fragments thereof being miR-133a, miR-10a, miR-34a, miR-32, miR191, miR885-5p, miR125a-5p, miR99b, miR146b-5p, miR-130a, miR-100, miR-130b, miR-486-3p, miR-500, miR128, miR-145, miR-221, miR-574-3p, miR-19a, miR-19b, miR-365, miR-345, miR-20a, miR-93, miR-20b, miR-223, miR-17, miR193a-5p, miR374b and miR-628-5p (i.e. sequences having at least 80% of sequence identity with SEQ ID NO: 3, 10, 14, 20, 46, 62, 66, 72, 75, 15, 17, 18, 19, 25, 26, 30, 39, 42, 44, 52, 53, 54, 55, 56, 63, 80, 81, 86, 88 and 91),
d) 17 distinct nucleic acid molecules, complements, equivalents and/or fragments thereof, said nucleic acid molecules, complements, equivalents and/or fragments thereof being hsa-miR-let-7d, hsa-miR-let-7e, hsa-miR-19a, hsa-miR-145, hsa-miR-191, hsa-miR-193a-5p, hsa-miR-195, hsa-miR-197, hsa-miR-221, hsa-miR-223, hsa-miR-365, hsa-miR-422a, hsa-miR-501-5p, hsa-miR-574-3p, hsa-miR-628-5p, hsa-miR-15b and hsa-miR-130a (i.e. sequences having at least 80% of sequence identity with SEQ ID NO: 49, 50, 53, 25, 75, 26, 77, 78, 80, 81, 30, 31, 40, 42, 44, 4 and 18 (table 8)),
e) 17 distinct nucleic acid molecules, complements, equivalents and/or fragments thereof, said nucleic acid molecules, complements, equivalents and/or fragments thereof being hsa-miR-let-7e, hsa-miR-let-7f hsa-miR-20a, hsa-miR-28-5p, hsa-miR-124, hsa-miR-126, hsa-miR-128, hsa-miR-132, hsa-miR-133b, hsa-miR-150, hsa-miR-191, hsa-miR-223, hsa-miR-342-3p, hsa-miR-365, hsa-miR-424, hsa-miR-218 and hsa-miR-449a (i.e. sequences having at least 80% of sequence identity with SEQ ID NO: 50, 2, 55, 9, 48, 67, 17, 68, 21, 73, 75, 81, 29, 30, 32, 28 and 33 (table 8)).
f) 6 distinct nucleic acid molecules, complements, equivalents and/or fragments thereof, said nucleic acid molecules, complements, equivalents and/or fragments thereof being hsa-miR-18b, hsa-miR-146-5p, hsa-miR-422a, hsa-miR-424, has-miR-138 and has-miR-218 (i.e. selected having at least 80% of sequence identity with SEQ ID NO: 5, 72, 31, 32, 22 and 28 (table 8)),
g) 8 distinct nucleic acid molecules, complements, equivalents and/or fragments thereof, said nucleic acid molecules, complements, equivalents and/or fragments thereof being hsa-miR-31, hsa-miR-99a, hsa-miR-126, hsa-miR-150, hsa-miR-125a-5p, hsa-miR-19b, hsa-miR-133b, hsa-miR-223 (i.e. sequences having at least 80% of sequence identity with sequences SEQ ID NO: 61, 13, 67, 73, 66, 54, 21 and 81 (table 8)),
h) 4 distinct nucleic acid molecules, complements, equivalents and/or fragments thereof, said nucleic acid molecules, complements, equivalents and/or fragments thereof being hsa-miR-31, hsa-miR-99a, hsa-miR-126, hsa-miR-150 (i.e. sequences having at least 80% of sequence identity with sequences SEQ ID NO: 61, 13, 67 and 73 (table 8)),
i) 3 distinct nucleic acid molecules, complements, equivalents and/or fragments thereof, said nucleic acid molecules, complements, equivalents and/or fragments thereof being hsa-miR-133b, hsa-miR-486-3p and hsa-miR-671-3p (i.e. sequences having at least 80% of sequence identity with sequences: SEQ ID NO: 21, 88 and 89 (table 8)),
j) two distinct nucleic acid molecules, complements, equivalents and/or fragments thereof, said nucleic acid molecules, complements, equivalents and/or fragments thereof being hsa-miR-376c and has-miR-885-5p (i.e. selected from sequences comprising or consisting of sequences having at least 80% of sequence identity with sequences selected from the group consisting of: SEQ ID NO: 47 and 46 (table 8)).

In any of the groups a), b), c), d), e), f), g), h), i), j) or k) defined above, the following additional nucleic acid molecules may further be used in addition of the ones identified above:

a) group a) further comprises an nucleic acid molecule, its complement, equivalent and/or fragment thereof, said nucleic acid molecule, complement, equivalent and/or fragment thereof comprising or consisting of miR-218 (i.e. said nucleic acid molecule comprising or consisting of a sequence having at least 80% of sequence identity with SEQ ID NO: 28),
c) group c) further comprises at least one nucleic acid molecule, complement, equivalent and/or fragment thereof, said nucleic acid molecule being selected from the group consisting of: miR-99a, miR-126, miR-150, miR422a, miR142-5p, miR-15b, miR-106b and miR-155. (i.e. said nucleic acid molecule being selected from sequences having at least 80% of sequence identity with sequences selected from the group consisting of: SEQ ID NO:13, 67, 73, 31, 24, 4, 65 and 84).

Another preferred diagnostic portfolio of the invention comprises more than one, more preferably at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 till 92 nucleic acid molecules, their complements, equivalents and/or fragments thereof as defined in the second diagnostic portfolio (i.e. sequences comprising or consisting of sequences having at least 80% of sequence identity with sequences selected from the group consisting of: SEQ ID NO: 49-140).

Therefore in an embodiment, a diagnostic portfolio comprises or consists of at least one or at least two or more nucleic acid molecules, their complements, equivalents, and/or fragments thereof, said nucleic acid molecules, complement, equivalent and/or fragment thereof being represented by the following sequences:
at least two sequences having each at least 80% of sequence identity with a sequence selected from the group consisting of SEQ ID NO:1-92 (table 3),
at least one sequence having at least 80% of sequence identity with a sequence selected from the group consisting of SEQ ID NO:29, 33, 43, 68, 71 and 90 (table 5, group a)
at least one sequence having at least 80% of sequence identity with a sequence selected from the group consisting of SEQ ID NO: 27, 34, 37, 45, 47, and 79 (table 6, group b),
at least one sequence having at least 80% of sequence identity with a sequence selected from the group consisting of: SEQ ID NO: 3, 10, 14, 20, 46, 62, 66, 72, 75 15, 17, 18, 19, 25, 26, 30, 39, 42, 44, 52, 53, 54, 55, 56, 63, 80, 81, 86, 88 and 91 (table 7, group c),
at least two sequences having each at least 80% of sequence identity with a sequence selected from the group consisting of: SEQ ID NO:49-140 (table 4),
at least one sequence comprising or consisting of sequences having at least 80% of sequence identity with sequences selected from the group consisting of: SEQ ID NO: 49, 50, 53, 25, 75, 26, 77, 78, 80, 81, 30, 31, 40, 42, 44, 4 and 18 (table 8), at least one sequence comprising or consisting of sequences having at least 80% of sequence identity with sequences selected from the group consisting of: SEQ ID NO: 50, 2, 55, 9, 48, 67, 17, 68, 21, 73, 75, 81, 29, 30, 32, 28 and 33 (table 8),
at least one sequence comprising or consisting of sequences having at least 80% of sequence identity with sequences selected from the group consisting of: SEQ ID NO: 5, 72, 31, 32, 22 and 28 (table 8),
at least one sequence comprising or consisting of sequences having at least 80% of sequence identity with sequences selected from the group consisting of: SEQ ID NO: 61, 13, 67, 73, 66, 54, 21 and 81 (table 8),
at least one sequence comprising or consisting of sequences having at least 80% of sequence identity with sequences selected from the group consisting of: SEQ ID NO: 61, 13, 67 and 73 (table 8), or
at least one sequence comprising or consisting of sequences having at least 80% of sequence identity with sequences selected from the group consisting of: SEQ ID NO: 21, 88 and 89 (table 8),
at least one sequence comprising or consisting of sequences having at least 80% of sequence identity with sequences selected from the group consisting of: SEQ ID NO: 47 and 46 (table 8)).

The invention allows the identification of nucleic acid molecules that are predictive for the presence, absence or susceptibility to inflammation. The invention therefore allows the assessment of the presence or absence of or the susceptibility to inflammation. Without wishing to be bound by any theory, it is believed that since these nucleic acid molecules were identified as having an expression which is modulated in a given group/phenotype of circulating CD14⁺ (first group) or CD4⁺ and CD8⁺ (second group) cells, these phenotype/group-associated nucleic acid molecules are believed to be strongly effective in predicting the presence or absence of or the susceptibility to inflammation in an individual or in a subject. Currently the most effective way of determining nucleic acid molecule expression level uses (micro)arrays.

Diagnostic portfolios comprising or consisting of any combinations or sub combinations as defined herein are also encompassed by the present invention.

A preferred diagnostic portfolio comprises a matrix suitable for identifying the differential expression of the nucleic acid molecules contained therein. A more preferred diagnostic portfolio comprises a matrix, wherein said matrix is employed in a microarray. Said microarray is preferably an oligonucleotide microarray.

There are at least two types of suitable matrices for the present invention: the solid matrix or solid support as such, and the solid support in solution. Examples of the second group are beads, e.g. magnetic beads or colour code beads. Colour code beads are preferred to be used in this invention. A preferred example of colour beads are the so-called xMAP beads of Luminex (see http://www.luminexcorp.com/TechnologiesScience/ for more info).

Kit

In a further aspect, there is provided an article including a representation of the nucleic acid molecule expression profiles that make up the portfolios useful for assessing the presence or absence of or the susceptibility to inflammation. These representations are reduced to a medium that can be automatically read by a machine such as computer readable media (magnetic, optical, and the like). The articles can also include instructions for assessing the nucleic acid molecule expression profiles in such media. For example, the articles may comprise a CD ROM having computer instructions for comparing nucleic acid molecule expression profiles of the portfolios of nucleic acid molecules described above. The articles may also have nucleic acid molecule expression profiles digitally recorded therein so that they may be compared with nucleic acid molecules expression data from a patient sample. Alternatively, the profiles can be recorded in different representational format. A graphical recordation is one such format.

Different types of articles of manufacture according to the invention are media or formatted assays used to reveal nucleic acid molecule expression profiles. Any of the nucleic acid sequence described herein may be comprised in a kit. These can comprise or consist of, for example, microarrays in which sequence complements or probes are affixed to a matrix to which the sequences indicative of the nucleic acid molecules combine creating a readable determinant of their presence. When such a microarray contains an optimized portfolio great savings in time, process steps, and resources are attained by minimizing the number of nucleic acid molecules that must be applied to the substrate, reacted with the sample, read by an analyser, processed for results, and (sometimes) verified.

Other articles according to the invention can be fashioned into reagent kits for conducting hybridization, amplification, and signal generation indicative of the level of expression of the nucleic acid molecules in the portfolios as defined herein. Kits made according to the invention include formatted assays for determining the nucleic acid molecule expression profiles. These can include all or some of the materials needed to conduct the assays such as reagents and instructions. Therefore, in a further aspect, there is provided a kit for assessing the presence or absence of or the susceptibility to inflammation in an individual comprising reagents for detecting nucleic acid sequences, complements, equivalents or fragments thereof, said nucleic acid sequences being represented by sequences comprising or consisting of sequences having at least 80% of sequence identity with SEQ ID NO:1-92, or 49-140 or any nucleic acid molecule derived thereof, preferably as identified above as a sub combination of at least one nucleic acid molecule identified in groups a), b), c), d), e), f), g), h), i) and/or j). Kits comprising or consisting of any combinations or sub combinations as defined herein are also encompassed by the present invention.

A preferred kit further comprises reagents for conducting a microarray analysis. More preferably, a kit further comprising a medium through which said nucleic acid sequences, complements, equivalents or fragments thereof are assayed. More preferably, said medium is a microarray.

A kit may also include instructions for employing the kit components as well the use of any other reagent not included in the kit. Instructions may include variations that can be implemented.

A kit may further include one or more negative control synthetic nucleic acid molecule such as miRNAs. A kit may further include water and hybridization buffer to facilitate hybridization of the two strands of the miRNAs.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit (labeling reagent and label may be packaged together), the kit also will generally contain a second, third or other additional container into which the additional components may be separately placed. However, various combinations of components may be comprised in a vial. The kits of the present invention also will typically include means for containing the nucleic acid molecules, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly preferred.

However, the components of the kit may be provided as dried powder(s). When reagents and/or components are provided as a dry powder, the powder can be reconstituted by the addition of a suitable solvent. It is envisioned that the solvent may also be provided in another container means.

Such kits may also include components that preserve or maintain a nucleic acid molecule or that protect against its degradation. Such components may be RNAse-free or protect against RNAses. Such kits generally will comprise, in suitable means, distinct containers for each individual reagent or solution.

Methods

In a further aspect, there is provided a method of assessing the presence or absence of or the susceptibility to inflammation in an individual using a diagnostic portfolio or a kit as earlier defined herein.

In another aspect, there is provided a method of assessing whether an individual responds to a given treatment by a decrease or delay or absence of inflammation using a diagnostic portfolio or a kit as earlier defined herein.

Each of these methods is preferably carried out ex vivo using a sample from the individual to be tested.

In a preferred embodiment, each of these methods comprises identifying differential modulation of a nucleic acid molecule present in said diagnostic portfolio (relative to the expression of a same nucleic acid molecule in a control).

In each of these methods, one may not try to identify a differential modulation of each of the nucleic acid molecules present in a diagnostic portfolio as earlier defined. One may try to identify a differential modulation in 5%, 10%, 20%, 30%, 40%, 50%, 60%, 70%, 80%, 90% or 100% of the nucleic acid molecules present in a diagnostic portfolio as identified herein. Preferred combinations of nucleic acid molecules have already been identified above.

In the context of the invention, “assessing the presence or absence of or the susceptibility to inflammation” means either a predictive risk assessment of inflammation in an individual (i.e. predict the presence of inflammation in the future, or pre-symptomatic prediction of risk of inflammation) or an assessment of the presence of inflammation in an individual.

In the context of the invention, “assessing whether an individual responds to a given treatment by a decrease or delay or absence of inflammation” may also refer to the likelihood that an individual will respond to a given therapy or to the response of an individual to a therapy he has already been administered. Such a method is crucial to have since, the therapy of said individual may be changed, adapted in order to obtain a better response to it.

In the context of the invention, an “individual” may be an animal or a human being. Preferably, an individual is a human being.

In the context of the invention, “inflammation” is being defined as a part of the biological response of an individual to a harmful stimulus such as a pathogen. Inflammation is a protective attempt by an individual to remove said stimuli and to initiate the healing process. In the context of the invention, “assessing the presence or absence of or the susceptibility to inflammation” may be used for predicting a given disease or condition which is associated with inflammation. Inflammation may be systemic or local inflammation. Inflammation may be chronic inflammation. Non-limiting examples of such diseases or conditions associated with inflammation are atherosclerosis, ischemic vascular disease, heart disease, cancer, chronic lower respiratory disease, stroke, rheumatoid arthritis, Alzheimer's disease, diabetes, allergy and nephritis, hypertension, hypercholesterolemia CAD, stable CAD versus unstable CAD, vessel disease including an increase of diseased coronary vessels and also including heart failure, atrial fibrillation, ischemic coronary and peripheral disease, claudicatio intemmittens. In a preferred embodiment, the invention is used for identify an individual at risk for the development of a cardiovascular disease.

Inflammation may be assessed using measurements of circulating biomarkers of inflammation such as soluble adhesion markers (e.g. E-selectin, P-selectin, intracellular adhesion molecule-1, vascular cell adhesion molecule-1), cytokines (e.g. interleukin-1β, interleukin-6, interleukin-8, interleukin 10, interleukin-12 or tumor necrosic factor-α), and acute phase reactants (fibrinogen, serum amyloid A protein and hi-sensitiviy C-reactive protein Hs-CRP). Hs-CRP is a stable serum marker that is used to classify an inflammatory state. In the context of the invention a control marker for inflammation is Hs-CRP: baseline, non-inflammatory states (<1.0 mg/L), intermediate inflammatory (1.0 to 3.0 mg/L) and inflammatory states (>3.0 mg/L) (Pearson et al, 2003) Hs-CRP may be first assessed in a sample from an individual. If the assessed concentration of Hs-CRP corresponds to an intermediate inflammatory to an inflammatory state, then the method of the invention could be applied to said individual to further assess the response and susceptibility of the circulating cells (CD14⁺′, CD4⁺ and/or CD8⁺) to this inflammatory state. Alternatively, the method of the invention may be first carried out without having first assessed the concentration of Hs-CRP. Hs-CRP reflects what the liver senses as pro-inflammatory molecules. It is expected that in the case of cardiovascular disease, the method of the invention is more specific than the assessment of Hs-CRP.

As indicated above, each of the methods of the invention comprises identifying differential modulation of a nucleic acid molecule present in said diagnostic portfolio. A nucleic acid molecule is preferably modulated when it is differentially expressed, i.e. up regulated (or increased or induced) or down regulated (or decreased) in CD14⁺ cells (i.e. monocytes) and/or in CD4⁺ and CD8⁺ cells by comparison to their expression in a corresponding control baseline. Up regulation and down regulation are relative terms meaning that a detectable difference (beyond the contribution of noise in the system used to measure it) is found in the expression of the nucleic acid molecules relative to a control baseline. In this case, a control baseline may come from a pool of CD14⁺ and/or CD4⁺ and/or CD8⁺ cells from a control individual which is known to be healthy. A control baseline may also be a pool of CD14⁺ and/or CD4⁺ and/or CD8⁺ cells from the same individual at the onset of a treatment or during a treatment. A pool of these (healthy) CD14⁺ and/or CD4⁺ and/or CD8⁺ cells preferably contains 1, 3, 5, 10, 20, 30, 100, 400, 500, 600 or more CD14⁺ and/or CD4⁺ and/or CD8⁺ cells obtained from at least one, 2, 5, 10 or more healthy individuals. The expression level of a nucleic acid molecule in the cells of the individual to be tested is then considered either up regulated or down regulated relative to a baseline level using the same measurement method. In an embodiment, the expression of a nucleic acid molecule as identified in a first diagnostic portfolio as earlier herein has been found up regulated (i.e. increased) or down regulated (i.e. decreased) in CD14⁺ cells of an individual to be tested by comparison to the expression of the same nucleic acid molecule in CD14⁺ cells of a control individual. Preferably, in this embodiment, the Hs-CRP concentration of said individual has been assessed as earlier indicated herein as a first step to assess the inflammatory status of said individual.

In another embodiment, the expression of a nucleic acid molecule as identified in a second diagnostic portfolio as earlier herein has been found up regulated (i.e. increased) or down regulated (i.e. decreased) in CD4⁺ and/or CD8⁺ cells of an individual to be tested by comparison to the expression of the same nucleic acid molecule in CD4⁺ and/or CD8⁺ cells of a control individual. Preferably, in this embodiment, the Hs-CRP concentration of said individual has been assessed as earlier indicated herein as a first step to assess the inflammatory status of said individual.

In the context of the use of diagnostic portfolios, a baseline is the measured nucleic acid molecule expression of a large pool of healthy CD14⁺, CD4⁺ and/or CD8⁺ cells from healthy individuals. Usually, large means at least 50 individuals, at least 70, 100, 150, 200, 250, 300, 350, 400, 450, 500, 550, 600 or more.

The assessment of the expression level of a nucleic acid molecule in order to assess whether said nucleic acid molecule is modulated is preferably performed using classical molecular biology techniques to detect mRNA levels, such as (real time) reverse transcriptase PCR (whether quantitative or semi-quantitative), mRNA (micro)array analysis or Northern blot analysis, or other methods to detect RNA. The skilled person will understand that alternatively or in combination with the quantification of an identified nucleic acid molecule, the quantification of a substrate of said corresponding nucleic acid molecule or of any compound known to be associated with the function of said corresponding nucleic acid molecule or the quantification of the function or activity of said corresponding nucleic acid molecule using a specific assay is encompassed within the scope of the method of the invention. In a preferred embodiment, the assessment of the expression level of a nucleic acid molecule is carried out using (micro)arrays as later defined herein.

Since the expression levels of a nucleic acid molecule may be difficult to be measured in an individual, a sample from said individual is preferably used. According to another preferred embodiment, the expression level of a nucleic acid molecule is determined ex vivo in a sample obtained from an individual. A sample may be liquid, semi-liquid, semi-solid or solid. A preferred sample comprises 100, 1000, 10000 or more CD14⁺ and/or CD4⁺ and/or CD8⁺ cells and/or a tissue from said individual to be tested taken in a biopsy. Alternatively and or in combination with earlier preferred embodiment, a sample preferably comprises or be derived from blood of an individual. The skilled person knows how to isolate and optionally purify RNA present in such a sample. In case of RNA, the skilled person may further amplify it using known techniques.

An increase (or up regulation) (which is synonymous with a higher expression level) or decrease (or down regulation) (which is synonymous with a lower expression level) of the expression level of a nucleic acid molecule is preferably defined as being a detectable change of the expression level of said nucleic acid molecule, equivalent or of a precursor thereof or any detectable change in a biological activity of said nucleic acid molecule or equivalent thereof using a method as defined earlier on as compared to the expression level of a corresponding nucleic acid molecule or equivalent or of a precursor thereof or the biological activity of said nucleic acid molecule or equivalent in a baseline. According to a preferred embodiment, an increase or decrease of a nucleic acid molecule activity is quantified using a specific assay for said activity.

Preferably, an increase of the expression level of a nucleic acid molecule (or equivalent thereof) or a precursor thereof means an increase of at least 5% of the expression level of said nucleic acid molecule (or equivalent thereof) or precursor thereof using arrays. More preferably, an increase of the expression level of said nucleic acid molecule (or equivalent thereof) or precursor thereof means an increase of at least 10%, even more preferably at least 20%, at least 30%, at least 40%, at least 50%, at least 70%, at least 90%, at least 150% or more.

Preferably, a decrease of the expression level of a nucleic acid molecule (or equivalent thereof) or a precursor thereof means a decrease of at least 5% of the expression level of said nucleic acid molecule (or equivalent thereof) or precursor thereof using arrays. More preferably, a decrease of the expression level of a nucleic acid molecule (or equivalent thereof) or a precursor thereof means a decrease of at least 10%, even more preferably at least 20%., at least 30%, at least 40%, at least 50%, at least 70%, at least 90%, at least 150% or more.

Preferably, an increase of a nucleic acid molecule (or equivalent thereof) activity means an increase of at least 5% of said activity using a suitable assay. More preferably, an increase of said activity means an increase of at least 10%, even more preferably at least 20%, at least 30%, at least 40%, at least 50%, at least 70%, at least 90%, at least 150% or more.

Preferably, a decrease of a nucleic acid molecule (or equivalent thereof) activity means a decrease of at least 5% of said activity using a suitable assay. More preferably, a decrease of said activity means a decrease of at least 10%, even more preferably at least 20%, at least 30%, at least 40%, at least 50%, at least 70%, at least 90%, at least 150% or more.

(Micro)arrays (or other high throughput screening devices) comprising a nucleic acid molecule as defined herein is a preferred way for carrying out a method of the invention. A microarray is a solid support or carrier containing one or more immobilised nucleic acid molecules for analysing nucleic acid sequences or mixtures thereof (see e.g. WO 97/27317, WO 97/22720, WO 97/43450, EP 0 799 897, EP 0 785 280, WO 97/31256, WO 97/27317, WO 98/08083 and Zhu and Snyder, 2001). (Micro)array technology allows for the measurement of the steady-state mRNA level of thousands of nucleic acid molecules simultaneously thereby presenting a powerful tool for identifying nucleic acid molecules modulation for a given group of nucleic acid molecules as identified herein. In a preferred embodiment, one uses an oligonucleotide array in a method of the invention. The product of an analyse is typically a measurement of the intensity of the signal received from a labelled probe used to detect a nucleic acid sequence from the sample that hybridizes to a nucleic acid sequence at a known location on the microarray. Typically, the intensity of the signal is proportional to the quantity of mRNA, expressed in a cell from an individual to be tested. A large number of such techniques are available and useful. Preferred methods for determining gene expression can be found in U.S. Pat. No. 6,271,002 to Linsley, et al.; U.S. Pat. No. 6,218,122 to Friend, et al.; U.S. Pat. No. 6,218,114 to Peck, et al.; and U.S. Pat. No. 6,004,755 to Wang, et al., the disclosure of each of which is incorporated herein by reference.

Analysis of the expression levels is conducted preferably by measuring expression levels using these techniques. Currently, this is best done by generating a matrix of the expression intensities of nucleic acid molecules in a test sample (RNA from cells from an individual to be tested) using a single channel hybridisation on a microarray platform, and comparing these intensities with the one of a reference group or baseline. It preferably means that within the context of the invention, a “control” refers to a large number of individuals as defined earlier herein preferably using the method as earlier defined herein.

Identification of a Substance Able to Prevent, Delay, Cure and/or Treat a Disease or a Condition Associated with Inflammation in an Individual

In yet a further aspect, the invention relates to a method for identification of a substance capable of preventing, delaying, curing and/or treating a disease or a condition associated with inflammation in an individual. The method preferably comprises the steps of: (a) providing a test cell population capable of expressing a nucleic acid molecule of the invention defined in the section entitled “diagnostic portfolio”; (b) contacting the test cell population with the substance; (c) determining the expression level of said nucleic acid molecule or an activity of said nucleic acid molecule in the test cell population contacted with the substance; (d) comparing the expression or activity level determined in (c) with the expression or activity level of said nucleic acid molecule in a test cell population that has not been contacted with the substance; and, (e) identifying a substance that produces a difference in expression level or activity level of said nucleic acid molecule, between the test cell population that has been contacted with the substance and the test cell population that has not been contacted with the substance.

Preferably, in step a), a test cell population is capable of expressing a nucleic acid molecule of the invention defined in the section entitled “diagnostic portfolio”. It is encompassed within the invention, that said test cell comprises a nucleic acid construct allowing the expression of a nucleic acid molecule as identified in the section entitled “diagnostic portfolio”. Preferably, in a method the expression or activity level of more than one nucleic acid molecule or precursor thereof is compared. Preferably, in a method, a test cell population comprises mammalian cells, more preferably human cells. Even more preferably, a test cell population comprises peripheral blood cells. These cells can be harvested, purified using techniques known to the skilled person. Even more preferably, a test cell population comprises a cell line. Preferably the cell line is a human cell line such as the myeloid cell line THP-1. In another preferred embodiment, test cells are part of an in vivo animal model. In one aspect the invention also pertains to a substance that is identified in a method the aforementioned methods.

In a preferred embodiment, “preventing” inflammation means that during at least one, two, three, four, five years, or longer no inflammation will be detected in an individual, wherein said individual is treated with said substance by comparison with a non-treated control.

In a preferred embodiment, “delaying” inflammation means that the detection of inflammation in an individual treated with said substance is delayed of at least 1, 6, 12, 18, 24, 30, 36, 42, 48, 54, 60, 66 months or longer compared to the time at which detection of inflammation will occur in a corresponding control non treated with said substance.

In a preferred embodiment, “treating”/“curing” inflammation means that there is a detectable decrease of inflammation in an individual treated with said substance after at least one month (2, 3, 4, 5, 6, 7, 8, 9, 10, 11, 12 months or longer) compared to the inflammation in the same individual before the onset of the treatment. A detectable decrease is preferably defined as being at least 1% decrease, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more till no inflammation is detectable.

In each of these embodiment, inflammation is preferably assessed as earlier defined herein.

### GENERAL DEFINITIONS AND GENERAL TECHNOLOGIES REFERRED TO HEREIN

MicroRNA molecules (“miRNAs”, “miRs” or “hsa-miRs”) are generally 21 to 22 nucleotides in length, though lengths of 17 and up to 25 nucleotides have been reported. Any length of 17, 18, 19, 20, 21, 22, 23, 24, 25 is therefore encompassed within the present invention and may be considered as a fragment of a miRNA molecule. The miRNAs are each processed from a longer precursor RNA molecule (“precursor miRNA”). Precursor miRNAs are transcribed from non-protein-encoding genes. A precursor may have a length of at least 50, 70, 75, 80, 85, 100, 150, 200 nucleotides ore more. The precursor miRNAs have two regions of complementarity that enables them to form a stem-loop- or fold-back-like structure, which is cleaved by enzymes called Dicer and Drosha in animals. Dicer and Drosha are ribonuclease Ill-like nucleases. The processed miRNA is typically a portion of the stem.

The processed miRNA (also referred to as “mature miRNA”) becomes part of a large complex, known as the RNA-Induced Silencing Complex (RISC) complex, to (down)-regulate a particular target gene. Examples of animal miRNAs include those that perfectly or imperfectly basepair with the mRNA target, resulting in either mRNA degradation or inhibition of translation respectively (Havelange V. et al, 2010) SiRNA molecules also are processed by Dicer, but from a long, double-stranded RNA molecule. SiRNAs are not naturally found in animal cells, but they can function in such cells in a RNA-induced silencing complex (RISC) to direct the sequence-specific cleavage of an mRNA target.

The study of endogenous miRNA molecules is described in U.S. Patent Application 60/575,743, which is hereby incorporated by reference in its entirety. A miRNA is apparently active in the cell when the mature, single-stranded RNA is bound by a protein complex that regulates the translation of mRNAs that hybridize to the miRNA. Introducing exogenous RNA molecules that affect cells in the same way as endogenously expressed miRNAs requires that a single-stranded RNA molecule of the same sequence as the endogenous mature miRNA be taken up by the protein complex that facilitates translational control. A variety of RNA molecule designs have been evaluated. Three general designs that maximize uptake of the desired single-stranded miRNA by the miRNA pathway have been identified. An RNA molecule with a miRNA sequence having at least one of the three designs may be referred to as a synthetic miRNA.

MiRNA Libraries

A key application for the miRNAs as identified herein is the assessment or diagnosis of the presence of one individual or groups of miRNAs in a sample. Cell populations with each of the different miRNAs can then be assayed to identify miRNAs whose presence reflects a cellular phenotype (i.e. inflammation). The number of different miRNAs in the libraries is variable. It is contemplated that there may be, be at least, or be at most 1, 2, 3, 4, 5, 6, 7, 8, 9, 10, 11, 12, 13, 14, 15, 16, 17, 18, 19, 20, 21, 22, 23, 25, 30, 35, 40, 45, 50, 55, 60, 65, 70, 75, 80, 85 or 90 or more, or any range derivable therein, different miRNA-specific molecules in the library. In specific embodiments, libraries have 1 to 20 or 5 to 40 or 10 to 90 different miRNA-specific molecules. “Different” miRNA-specific molecules refers to nucleic acids that specifically encode miRNAs with different sequences. The wording “sequences selected from the group consisting of” indicates that any combination of sequences present in that group is encompassed by the present invention. This expression may be replaced by the expression “sequences selected from”.

A preferred diagnostic portfolio or library of the invention comprises more than one, more preferably at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 till 92 nucleic acid molecules, their complement and/or fragments thereof as defined in the first diagnostic portfolio (i.e. sequences comprising or consisting of sequences having at least 80% of sequence identity with sequences selected from the group consisting of SEQ ID NO: 1-92 or of at least one sequences identified in each of
group a: SEQ ID NO: 29, 33, 43, 68, 71 and 90 (table 5),
group b: SEQ ID NO: 27, 34, 37, 45, 47, and 79 (table 6),
group c: SEQ ID NO: 3, 10, 14, 20, 46, 62, 66, 72, 75, 15, 17, 18, 19, 25, 26, 30, 39, 42, 44, 52, 53, 54, 55, 56, 63, 80, 81, 86, 88 and 91 (table 7).
group d: SEQ ID NO: 49, 50, 53, 25, 75, 26, 77, 78, 80, 81, 30, 31, 40, 42, 44, 4 and 18 (table 8),
group e: SEQ ID NO: 50, 2, 55, 9, 48, 67, 17, 68, 21, 73, 75, 81, 29, 30, 32, 28 and 33 (table 8),
group f: SEQ ID NO: 5, 72, 31, 32, 22 and 28 (table 8),
group g: SEQ ID NO: 61, 13, 67, 73, 66, 54, 21 and 81 (table 8),
group h: SEQ ID NO: 61, 13, 67 and 73 (table 8),
group i: SEQ ID NO: 21, 88 and 89 (table 8), and
group j: SEQ ID NO: 47 and 46.

Another preferred diagnostic portfolio or library of the invention comprises more than one, more preferably at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91 till 92 nucleic acid molecules, their complement and/or fragments thereof as defined in the second diagnostic portfolio (i.e. sequences comprising or consisting of sequences having at least 80% of sequence identity with sequences selected from the group consisting of SEQ ID NO: 49-140).

miRNAs are contemplated to be made primarily of RNA, though in some embodiments, they may be RNA, nucleotide analogs, such as Locked nucleic acids (LNA) or Unlocked nucleic acids (UNA), DNA, or any combination of DNA, RNA, nucleotide analogs, and PNAs (Peptide Nucleic Acids). Accordingly, it is understood that the library contains one or more nucleic acids for these different miRNAs. In specific embodiments, the library is specific to human miRNAs, though libraries for multiple organisms are contemplated.

An RNA molecule of the invention has or comprises or consists of a miRNA region. In specific embodiments, a miRNA molecule or equivalent thereof has a sequence that derives from any of SEQ ID NOs: 1-92 or 49-140 (Tables 3, 4). It is particularly contemplated that nucleic acid molecules of the invention may be derived from any of the mature miRNA sequences in SEQ ID NOs: 1-92 or 49-140.

A miRNA molecule or equivalent thereof will include a sequence that extends at least 1 to 5 nucleotides of coding sequence upstream and/or downstream of the predicted miRNA sequence. In some embodiments, molecules have up to 1, 2, 3, 4, 5, 6, 7, or more contiguous nucleotides, or any range derivable therein, that flank the sequence encoding the predominant processed miRNA on one or both sides (5′ and/or 3′ end).

Libraries or portfolio of the invention can contain miRNA sequences from any organism having miRNAs, specifically including but not limited to, mammals such as humans, non human primates, rats and mice. Specifically contemplated are libraries having, having at least, or having at most 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85 or 90 or more different miRNAs (that is, miRNA-specific molecules having different sequences derived from different miRNA genes). Specifically contemplated are such libraries described in the previous sentence with respect to any of SEQ ID NOs: 1-92 or 49-140 particularly those corresponding to miRNA sequences (mature sequence).

The term “miRNA” or “miR” or “hsa-miR” generally refers to a single-stranded molecule, but in specific embodiments, molecules implemented in the invention will also encompass a region or an additional strand that is partially (between 10 and 50% complementary across length of strand), substantially (greater than 50% but less than 100% complementary across length of strand) or fully complementary to another region of the same single-stranded molecule or to another nucleic acid. Thus, nucleic acids may encompass a molecule that comprises one or more complementary or self-complementary strand(s) or “complement(s)” of a particular sequence comprising a molecule. For example, precursor miRNA may have a self-complementary region, which is up to 100% complementary.

Within the whole text of the application unless otherwise indicated, a miRNA as for example present in a diagnostic portfolio of the invention may also be named a miRNA molecule, a miR, or an equivalent thereof. Each sequence identified herein may be identified as being SEQ ID NO as used in the text of the application or as corresponding SEQ ID NO in the sequence listing. A nucleic acid molecule, a miRNA molecule or an equivalent or a fragment thereof may be considered as an isolated nucleic acid molecule, an isolated miRNA molecule or an isolated equivalent or an isolated fragment thereof.

Preferably a miRNA molecule or an equivalent thereof is from 6 to 30 nucleotides in length, preferably 12 to 30 nucleotides in length, preferably 15 to 28 nucleotides in length, more preferably said molecule has a length of at least 6, 7, 8, 9, 10, 11, 12, 13, 14, 15, 16, 17, 18, 19, 20, 21, 22, 23, 24, 25, 26, 27, 28, 29, 30 nucleotides or more.

In a preferred embodiment, a miRNA molecule or equivalent thereof comprises at least 6 of the 7 nucleotides present in the seed sequence of said miRNA molecule or equivalent thereof

In another preferred embodiment, a miRNA molecule or equivalent sequence as encompassed by the present invention is as identified in Table 3, 4, 5, 6 or 7 as SEQ ID NO:1-140 or any sequence having at least 65% identity with SEQ ID NO:1-140. may have at least 80% identity with SEQ ID NO:1-140. Identity may be at least 65%, 70%, 75%, 80%, 85%, 90%, 95%, 99% or 100%. Identity is preferably assessed on the whole SEQ ID NO as identified in a given Table. However, identity may also be assessed on part of a given SEQ ID NO. Part may mean at least 50% of the length of the SEQ ID NO, at least 60%, at least 70%, at least 80%, at least 90% or 100%.

As used herein, “hybridization”, “hybridizes” or “capable of hybridizing” is understood to mean the forming of a double or triple stranded molecule or a molecule with partial double or triple stranded nature using techniques known to the skilled person such as southern blotting procedures. The term “anneal” as used herein is synonymous with “hybridize.” The term “hybridization”, “hybridize(s)” or “capable of hybridizing” may mean “low”, “medium” or “high” hybridization conditions as defined below. Low to medium to high stringency conditions means prehybridization and hybridization at 42° C. in 5×SSPE, 0.3% SDS, 200 pg/ml sheared and denatured salmon sperm DNA, and either 25% 35% or 50% formamide for low to medium to high stringencies respectively. Subsequently, the hybridization reaction is washed three times for 30 minutes each using 2×SSC, 0.2% SDS and either 55° C., 65° C., or 75° C. for low to medium to high stringencies.

Nucleic acids or derivatives thereof of the invention will comprise, in some embodiments the miRNA sequence of any miRNA described in SEQ ID NOs: 1-92 or 49-140. It is contemplated that nucleic acids sequences of the invention derived from SEQ ID NO:1-92 or 49-140 can have, have at least, or have at most 5, 6, 7, 8, 9, 10, 11, 12, 13, 14, 15, 16, 17, 18, 19, 20, 21, 22, 23, contiguous nucleotides from SEQ ID NOs: 1-92 or 49-140 (or any range derivable therein). In other embodiments, nucleic acids are, are at least, or are at most 80, 81, 82, 83, 84, 85, 86, 87, 88, 89, 90, 91, 92, 93, 94, 95, 96, 97, 98, 99, 100% identical to the miRNA sequence of SEQ ID NOs: 1-92 or 49-140 or to the precursor sequence of any of SEQ ID NO: 1-92 or 49-140 or any combination or range derivable therein.

Labeling and Labeling Techniques

In some embodiments, the present invention concerns miRNAs that are labeled, such as for screening assays to evaluate the therapeutic or diagnostic relevance of a particular miRNA species. It is contemplated that miRNA may first be isolated (either from a cell in which the miRNA is endogenous to the cell or from a cell in which miRNA is exogenous to the cell) and/or purified prior to labeling. This may be achieved by a reaction that more efficiently labels the miRNA, as opposed to other RNA in a sample in which the miRNA is not isolated or purified prior to labeling. In many embodiments of the invention, the label is non-radioactive. Generally, nucleic acids may be labeled by adding labeled nucleotides (one-step process) or adding nucleotides and labeling the added nucleotides (two-step process).

Moreover, miRNAs may be labeled as is described in U.S. Patent Application Ser. No. 60/649,584, which is hereby incorporated by reference. Such nucleotides include those that can be labeled with a dye, including a fluorescent dye, or with a molecule such as biotin. Labeled nucleotides are readily available; they can be acquired commercially or they can be synthesized by reactions known to those of skill in the art.

Nucleotides for Labeling

Nucleotides for labelling are not naturally occurring nucleotides, but instead, refer to prepared nucleotides that have a reactive moiety on them. Specific reactive functionalities of interest include: amino, sulfhydryl, sulfoxyl, aminosulfhydryl, azido, epoxide, isothiocyanate, isocyanate, anhydride, monochlorotriazine, dichlorotriazine, mono- or dihalogen substituted pyridine, mono- or disubstituted diazine, maleimide, epoxide, aziridine, sulfonyl halide, acid halide, alkyl halide, aryl halide, alkylsulfonate, N-hydroxysuccinimide ester, imido ester, hydrazine, azidonitrophenyl, azide, 3-(2-pyridyl dithio)-propionamide, glyoxal, aldehyde, iodoacetyl, cyanomethyl ester, p-nitrophenyl ester, o-nitrophenyl ester, hydroxypyridine ester, carbonyl imidazole, and the other such chemical groups. In some embodiments, the reactive functionality may be bonded directly to a nucleotide, or it may be bonded to the nucleotide through a linking group. The functional moiety and any linker cannot substantially impair the ability of the nucleotide to be added to the miRNA or to be labeled. Representative linking groups include carbon containing linking groups, typically ranging from about 2 to 18, usually from about 2 to 8 carbon atoms, where the carbon containing linking groups may or may not include one or more heteroatoms, e.g. S, O, N etc., and may or may not include one or more sites of unsaturation. Of particular interest in many embodiments are alkyl linking groups, typically lower alkyl linking groups of 1 to 16, usually 1 to 4 carbon atoms, where the linking groups may include one or more sites of unsaturation. The functionalized nucleotides (or primers) used in the above methods of functionalized target generation may be fabricated using known protocols or purchased from commercial vendors, e.g., Sigma, Roche, Ambion, and IDT. Functional groups may be prepared according to ways known to those of skill in the art, including the representative information found in U.S. Pat. Nos. 4,404,289; 4,405,711; 4,337,063 and 5,268,486, and Br. Pat. No. 1,529,202, which are all incorporated by reference.

Amine-modified nucleotides are used in several embodiments of the invention. The amine-modified nucleotide is a nucleotide that has a reactive amine group for attachment of the label. It is contemplated that any ribonucleotide (G, A, U, or C) or deoxyribonucleotide (G, A, T, or C) can be modified for labeling. Examples include, but are not limited to, the following modified ribo- and deoxyribo-nucleotides: 5-(3-aminoallyl)-UTP; 8-[(4-amino)butyl]-amino-ATP and 8-[(6-amino)butyl]-amino-ATP; N⁶-(4-amino)butyl-ATP, N⁶-(6-amino)butyl-ATP, N⁴-[2,2-oxy-bis-(ethylamine)]-CTP; N⁶-(6-Amino)hexyl-ATP; 8-[(6-Amino)hexyl]-amino-ATP; 5-propargylamino-CTP, 5-propargylamino-UTP; 5-(3-aminoallyl)-dUTP; 8-[(4-amino)butyl]-amino-dATP and 8-[(6-amino)butyl]-amino-dATP; N-(4-amino)butyl-dATP, N⁶-(6-amino)butyl-dATP, N⁴[2,2-oxy-to-(ethylamine)]-dCTP; N⁶-(6-Amino)hexyl-dATP; 8-[(6-Amino)hexyl]-amino-dATP; 5-propargylamino-dCTP, and 5-propargylamino-dUTP. Such nucleotides can be prepared according to methods known to those of skill in the art. Moreover, a person of ordinary skill in the art could prepare other nucleotide entities with the same amine-modification, such as a 5-(3-aminoallyl)-CTP, GTP, ATP, dCTP, dGTP, dTTP, or dUTP in place of a 5-(3-aminoallyl)-UTP.

Labeling Techniques

In some embodiments, nucleic acids are labeled by catalytically adding to the nucleic acid an already labeled nucleotide or nucleotides. One or more labeled nucleotides can be added to miRNA molecules. See U.S. Pat. No. 6,723,509, which is hereby incorporated by reference.

In other embodiments, an unlabeled nucleotide or nucleotides is catalytically added to an miRNA, and the unlabeled nucleotide is modified with a chemical moiety that enables it to be subsequently labeled, in embodiments of the invention, the chemical moiety is a reactive amine such that the nucleotide is an amine-modified nucleotide. Examples of amine-modified nucleotides are well known to those of skill in the art, many being commercially available such as from Ambion, Sigma, Jena Bioscience, and TriLink.

In contrast to labeling of cDNA during its synthesis, the issue for labeling miRNAs is how to label the already existing molecule. To this end, we may use an enzyme capable of using a di- or tri-phosphate ribonucleotide or deoxyribonucleotide as a substrate for its addition to an miRNA, a small RNA molecule. Moreover, in specific embodiments, it involves using a modified di- or triphosphate ribonucleotide, which is added to the 3′ end of an miRNA. The source of the enzyme is not limiting. Examples of sources for the enzymes include yeast, gram-negative bacteria such as E. coli, lactococcus lactis, and sheep pox virus.

Enzymes capable of adding such nucleotides include, but are not limited to, poly(A) polymerase, terminal transferase, and polynucleotide phosphorylase. In specific embodiments of the invention, ligase is contemplated as not being the enzyme used to add the label, and instead, a non-ligase enzyme is employed.

Poly(A) polymerase has been cloned from a number of organisms from plants to humans. It has been shown to catalyze the addition of homopolymer tracts to RNA (Martin et al, RNA, 4(2):226-30, 1998).

Terminal transferase catalyzes the addition of nucleotides to the 3′ terminus of a nucleic acid.

Polynucleotide phosphorylase can polymerize nucleotide diphosphates without the need for a primer.

Labels and Tags

miRNAs or miRNA probes may be labeled with a positron emitting (including radioactive), enzymatic, colorimetric (includes visible and UV spectrum, including fluorescent), luminescent or other label or tag for detection or isolation purposes. The label may be detected directly or indirectly. Radioactive labels include ¹²⁵I, ³²P, ³³P, and ³⁵S. Examples of enzymatic labels include alkaline phosphatase, luciferase, horseradish peroxidase, and β-galactosidase. Labels can also be proteins with luminescent properties, e.g., green fluorescent protein and phicoerythrin.

The colorimetric and fluorescent labels contemplated for use as conjugates include, but are not limited to, AMCA, Alexa Fluor dyes, BODIPY dyes, such as BODIPY FL, BODIPY 630/650, BODIPY 650/665, BODIP Y-R6G, BODIPY-TRX; Cascade Blue; Cascade Yellow; coumarin and its derivatives, such as 7-amino-4-methylcoumarin, aminocoumarin and hydroxycoumarin; cyanine dyes, such as Cy3 and Cy5; eosins and erythrosins; fluorescein and its derivatives, such as fluorescein isothiocyanate; macrocyclic chelates of lanthanide ions, such as Quantum Dye™; Marina Blue; Oregon Green; rhodamine dyes, such as rhodamine red, tetramethylrhodamine and rhodamine 6G; Texas Red;

Specific examples of dyes include, but are not limited to, those identified above and the following: Alexa Fluor 350, Alexa Fluor 405, Alexa Fluor 430, Alexa Fluor 488, Alexa Fluor 500. Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, and, Alexa Fluor 750; amine-reactive BODIPY dyes, such as BODIPY 493/503, BODEPY 530/550, BODEPY 558/568, BODIPY 564/570, BODDPY 576/589, BODIPY 581/591, BODEPY 630/650, BODIPY 650/655, BODIPY FL, BODIPY R6G, BODEPY TMR, and, BODIPY-TR; Cy3, Cy5, 6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, 2′,4′,5′,7′-Tetrabromosulfonefluorescein, and TET.

Specific examples of fluorescently labeled ribonucleotides are available from Molecular Probes, and these include, Alexa Fluor 488-5-UTP, Fluorescein-12-UTP, BODEPY FL-14-UTP, BODIPY TMR-14-UTP, Tetramethylrhodamine-6-UTP, Alexa Fluor 546-14-UTP, Texas Red-5-UTP, and BODIPY TR-14-UTP. Other fluorescent ribonucleotides are available from Amersham Biosciences, such as Cy3-UTP and Cy5-UTP. Examples of fluorescently labeled deoxyribonucleotides include Dinitrophenyl (DNP)-11-dUTP, Cascade Blue-7-dUTP, Alexa Fluor 488-5-dUTP, Fluorescein-12-dUTP, Oregon Green 488-5-dUTP, BODEPY FL-14-dUTP, Rhodamine Green-5-dUTP, Alexa Fluor 532-5-dUTP, BODEPY TMR-14-dUTP, Tetramethylrhodamine-6-dUTP, Alexa Fluor 546-14-dUTP, Alexa Fluor 568-5-dUTP, Texas Red-12-dUTP, Texas Red-5-dUTP, BODEPY TR-14-dUTP, Alexa Fluor 594-5-dUTP, BODEPY 630/650-14-dUTP, BODIPY 650/665-14-dUTP; Alexa Fluor 488-7-OBEA-dCTP, Alexa Fluor 546-16-OBEA-dCTP, Alexa Fluor 594-7-OBEA-dCTP, Alexa Fluor 647-12-OBEA-dCTP. It is contemplated that nucleic acids may be labeled with two different labels. Furthermore, fluorescence resonance energy transfer (FRET) may be employed in methods of the invention (e.g., Klostermeier et al., 2002; Emptage, 2001; Didenko, 2001, each incorporated by reference). Fluorescent energy transfer dyes, such as thiazole orange-ethidium heterodimer; and, TOTAB may be used.

Alternatively, the label may not be detectable per se, but indirectly detectable or allowing for the isolation or separation of the targeted nucleic acid. For example, the label could be biotin, digoxigenin, polyvalent cations, chelator groups and the other ligands, include ligands for an antibody.

Visualization Techniques

A number of techniques for visualizing or detecting labeled nucleic acids are readily available. The reference by Stanley T. Crooke, 2000 has a discussion of such techniques (Chapter 6), which is incorporated by reference. Such techniques include, microscopy, arrays, Fluorometry, Light cyclers or other real time PCR™ machines, FACS analysis, scintillation counters, Phosphoimagers, Geiger counters, MM, CAT, antibody-based detection methods (Westerns, immunofluorescence, immunohistochemistry), histochemical techniques, HPLC (Griffey et al, 1997, spectroscopy, capillary gel electrophoresis (Cummins et ah, 1996), spectroscopy; mass spectroscopy; radiological techniques; and mass balance techniques. Alternatively, nucleic acids may be labeled or tagged to allow for their efficient isolation. In other embodiments of the invention, nucleic acids are biotinylated.

When two or more differentially colored labels are employed, fluorescent resonance energy transfer (FRET) techniques may be employed to characterize the dsRNA. Furthermore, a person of ordinary skill in the art is well aware of ways of visualizing, identifying, and characterizing labeled nucleic acids, and accordingly, such protocols may be used as part of the invention. Examples of tools that may be used also include fluorescent microscopy, a BioAnalyzer, a plate reader, Storm (Molecular Dynamics), Array Scanner, FACS (fluorescent activated cell sorter), or any instrument that has the ability to excite and detect a fluorescent molecule (Acumen [TTP Labtech] plate cytometer for example.

Array Preparation

The present invention can be employed with miRNA arrays, which are ordered macroarrays or microarrays of nucleic acid molecules (probes) that are fully or nearly complementary or identical to a plurality of miRNA molecules or precursor miRNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of miRNA-complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 806; 5,525,464; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference. It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments, hi certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO03067217; WO 03066906; WO 03076928; WO 03093810; WO 03100448A1, all of which are specifically incorporated by reference.

Recently, alternative profiling methods have become available, based on solution hybridization and subsequent immobilization and identification e.g. Illumina platform.

Sample Preparation

It is contemplated that the miRNA of a wide variety of samples can be analyzed using assays described herein. While endogenous miRNA is contemplated for use with some embodiments, recombinant or synthetic miRNA—including nucleic acids that are identical to endogenous miRNA or precursor miRNA—can also be handled and analyzed as described herein. Samples may be biological samples, in which case, they can be from blood, CSF, tissue, organs, tumor, semen, sputum, stool, urine, saliva, tears, other bodily fluid, hair follicles, skin, or any sample containing or constituting biological cells. Alternatively, the sample may not be a biological sample, but be a chemical mixture, such as a cell-free reaction mixture (which may contain one or more biological enzymes).

### Nucleic Acid Molecule Defined by a SEQ ID NO and Sequence Identity

It is to be understood that each nucleic acid molecule as identified herein by a given Sequence Identity Number (SEQ ID NO) is not limited to this specific sequence as disclosed. Each nucleic acid sequence or nucleotide sequence as identified herein encodes a given nucleic acid molecule identified in tables 3, 4, 5, 6 and 7. Throughout this application, each time one refers to a nucleic acid molecule, one may replace it by a corresponding nucleotide or nucleic acid sequence SEQ ID NO, if we take SEQ ID NO:X as example, one may replace it by:
i. a nucleic acid sequence that has at least 80% sequence identity with a nucleic acid sequence SEQ ID NO:X as identified in tables 3-7,
ii a fragment of SEQ ID NO:X
iii. a nucleic acid sequence the complementary strand of which hybridizes to a nucleic acid molecule of sequence of (i),

A fragment is defined as 50%, 60%, 70%, 80%, 90%, 100% of the length of the corresponding SEQ ID NO:X.

Each nucleotide or nucleic acid sequence described herein by virtue of its identity percentage (at least 80%) with a given nucleotide or nucleic sequence respectively has in a further preferred embodiment an identity of at least 80%, 85%, 90%, 95%, 97%, 98%, 99% or more identity with the given nucleotide or nucleic acid sequence. In a preferred embodiment, sequence identity is determined by comparing the whole length of the sequences as identified herein. Identity may alternatively be assessed on a part of a sequence. Part may mean at least 50%, 60%, 70%, 80%, 90% or 100% of the length of said sequence.

“Sequence identity” is herein defined as a relationship between two or more nucleic acid (polynucleotide) sequences, as determined by comparing the sequences. In the art, “identity” also means the degree of sequence relatedness between nucleic acid sequences, as the case may be, as determined by the match between strings of such sequences. “Identity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a diagnostic portfolio, a kit or a method as defined herein may comprise additional component(s), respectively additional step(s) than the ones specifically identified, said additional component(s), respectively said additional step(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent and literature references cited in the present specification are hereby incorporated by reference in their entirety.

The invention is further illustrated by the following examples which should not be construed for limiting the scope of the present invention.

### DESCRIPTION OF THE FIGURES

FIG. 1. The impact of chronic inflammation on the hematopoietic cells involved in vascular maintenance and repair. Cardiovascular risk factors almost without exception lead to elevated chronic systemic inflammation. This condition impacts on the myeloid progenitors and “SKEW” these cells into a more inflammatory differentiation pathway. As a consequence, monocytes in the circulation display the phenotype of a pre-mature CD16⁺ macrophage. This happens at the cost of the potential of early monocytes to adopt an regenerative pro-angiogenic mEPC like phenotype. The loss of mEPC impairs the vascular regenerative capacity and the increase in the number and function of the pro-inflammatory myeloid cells contributes to the development of atherosclerotic vascular lesions. Phenotypic alterations of the circulating CD14 positive myeloid cells can be assessed by their microRNAs and reflect the relative risk of the individual for the development of cardiovascular disease

FIG. 2. Schematic representation of the strategy used to select CD14⁺, CD4⁺ and CD8⁺ phenotype specific miRNAs

FIG. 2A. Outline of the strategy to select microRNAs that are associated with phenotypic changes in circulating myeloid cells in coronary artery disease (CAD) versus healthy subjects. To generate a maximally broad spectrum of miRNA expression profiles from CD14⁺-derived cells ranging from pro-inflammatory M1 macrophages to the reparative M2 macrophages and myeloid angiogenic accessory cells (mEPC), CD14⁺ cells were isolated from healthy control subjects and cultured as described in the text. In addition, to assess the spectrum of microRNAs that are differentially expressed in circulating CD14⁺ cells in patients with established coronary artery disease compared to healthy control subjects we isolated CD14⁺ cells from 6 CAD patients and 6 healthy controls. From all cells types total RNA was isolated and prepared for Solid deep sequencing and megaplex profiling. From the acquired miRNA expression sets 92 miRNAs were selected (described) that were most potent to detect changes in the phenotype of circulating CD14⁺ cells.

FIG. 2B. Outline of the strategy to select microRNA that are associated with phenotypic changes in circulating CD4⁺ and CD8⁺ cells in coronary artery disease versus healthy subjects. To generate a broad spectrum of miRNA expression profiles from CD4⁺ cell we isolated fresh CD4⁺ from healthy control subjects and cultured these cells to TH1 and TH2 cells as described in the text. In addition, to assess the spectrum of microRNAs that are differentially expressed in circulating CD4⁺ and CD8⁺ cells in patients with established coronary artery disease compared to healthy control subjects we isolated CD4⁺ and CD8⁺ cells from 6 CAD patients and 6 healthy controls. From all cells types total RNA was isolated and prepared for Solid deep sequencing and megaplex profiling. From the acquired miRNA expression sets 92 miRNAs were selected (described) that were most useful to detect changes in the phenotype of circulating CD4⁺ and CD8⁺ cells.

FIG. 3. Flow sheet selection CD14⁺ cell phenotype selective miRNAs

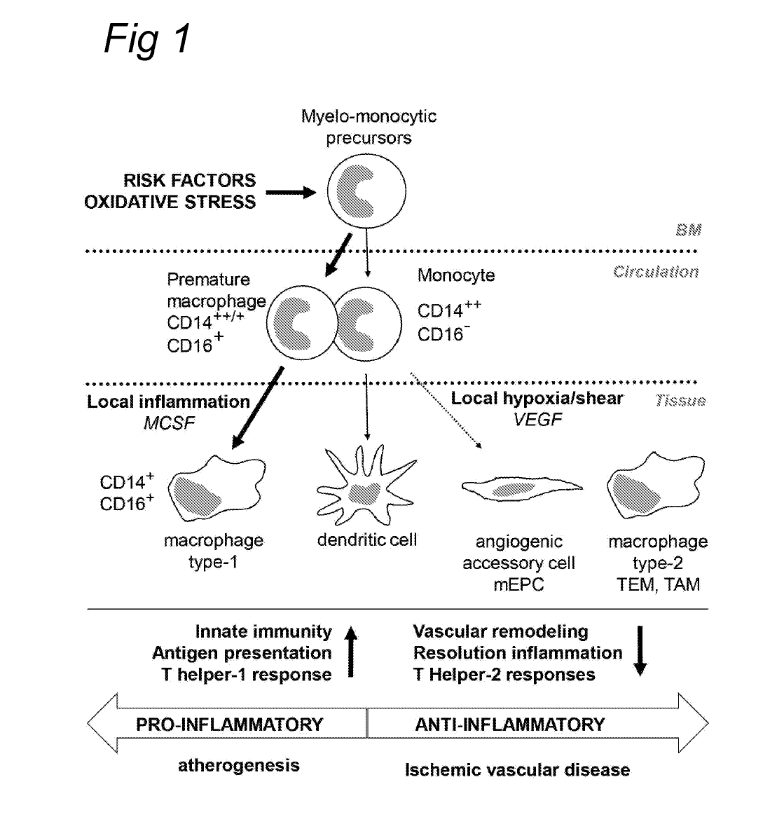

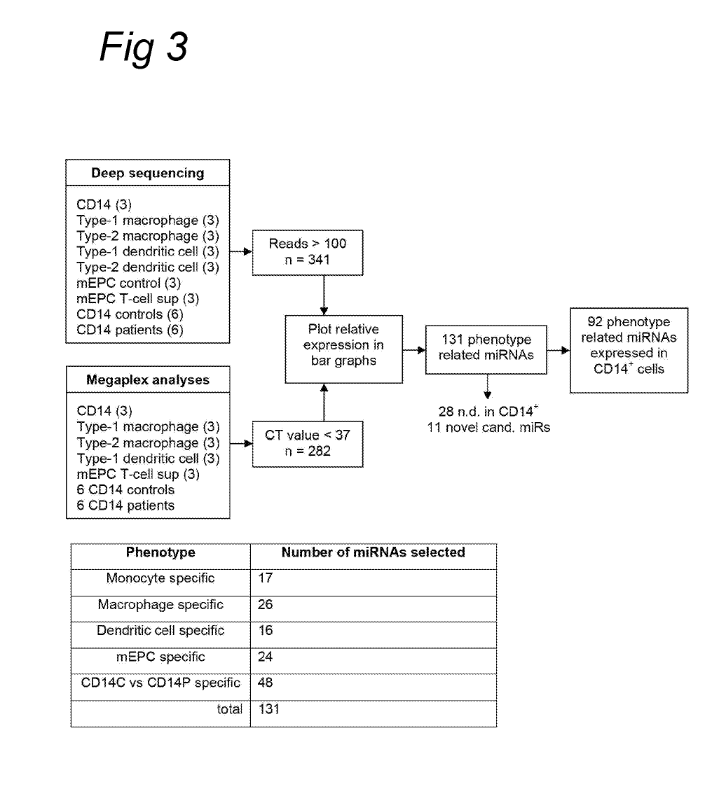

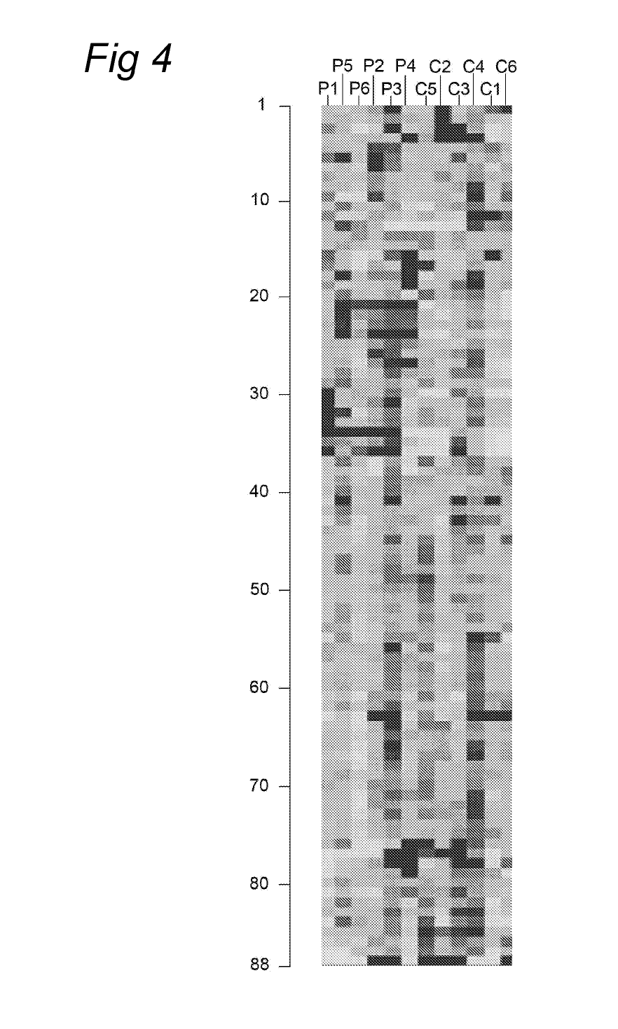

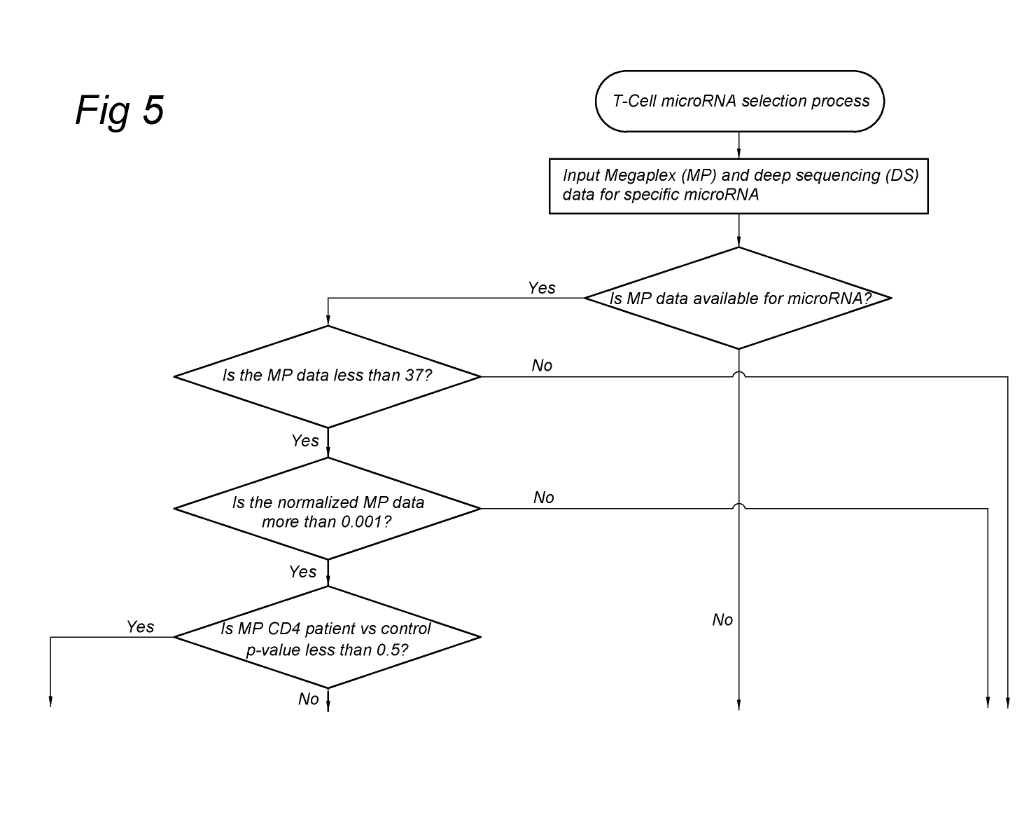

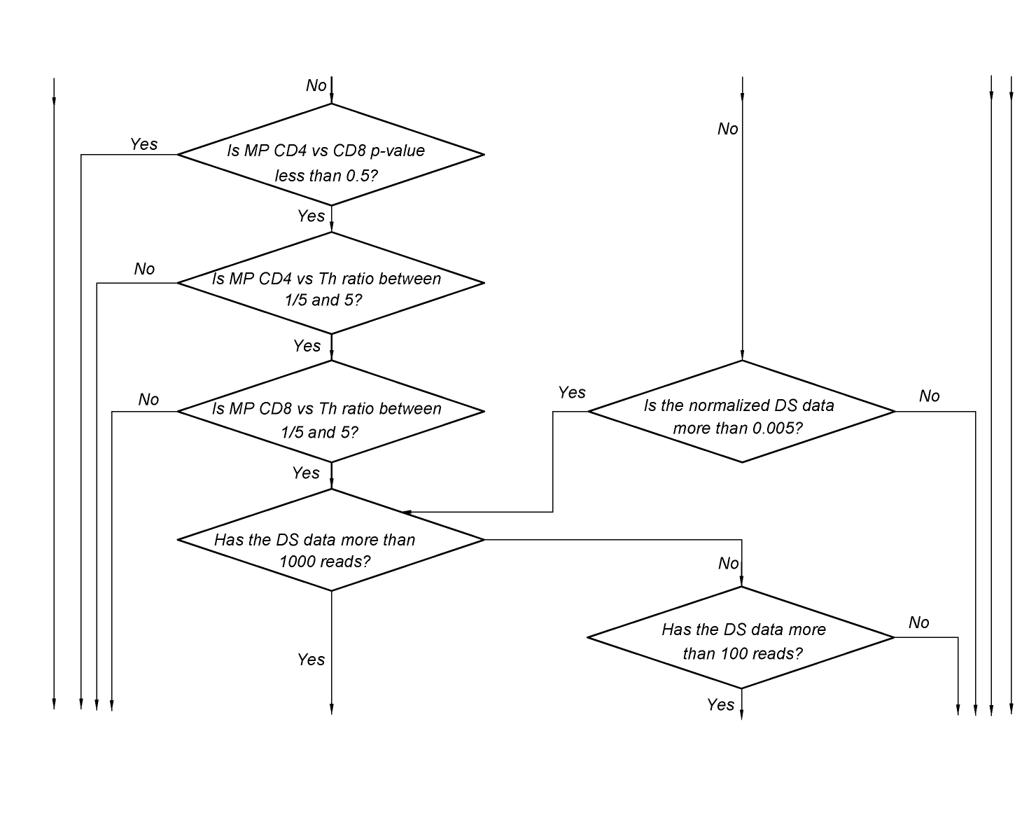

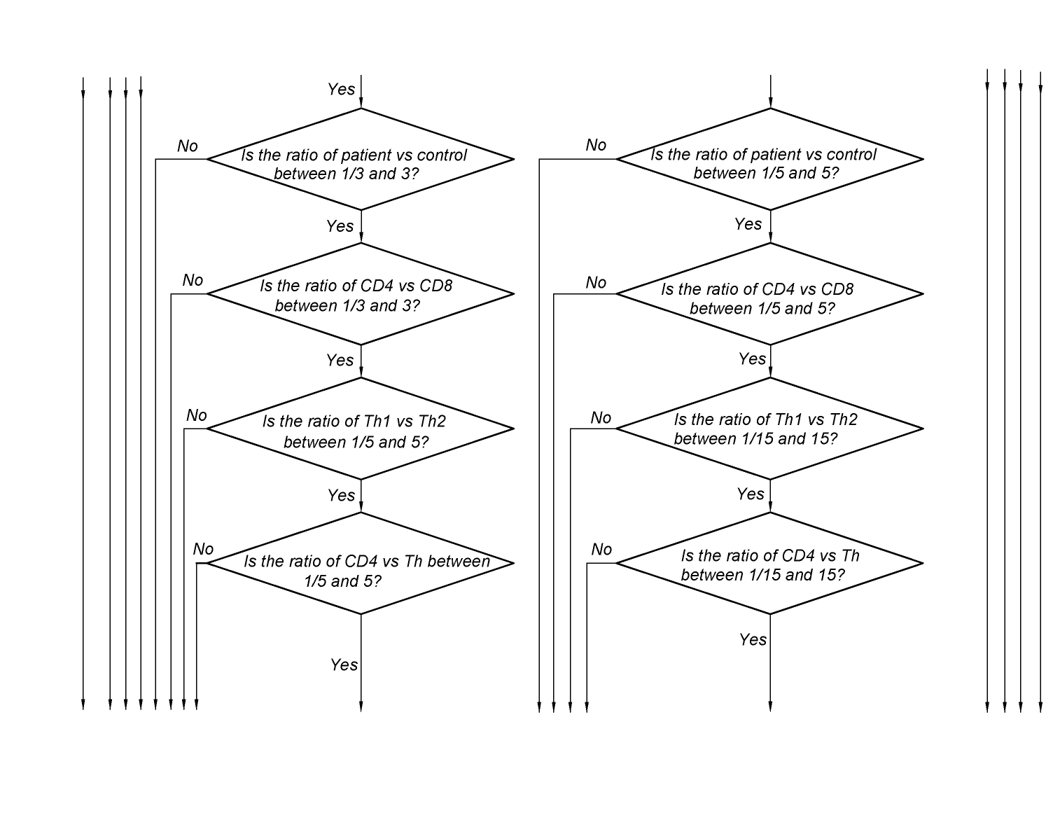

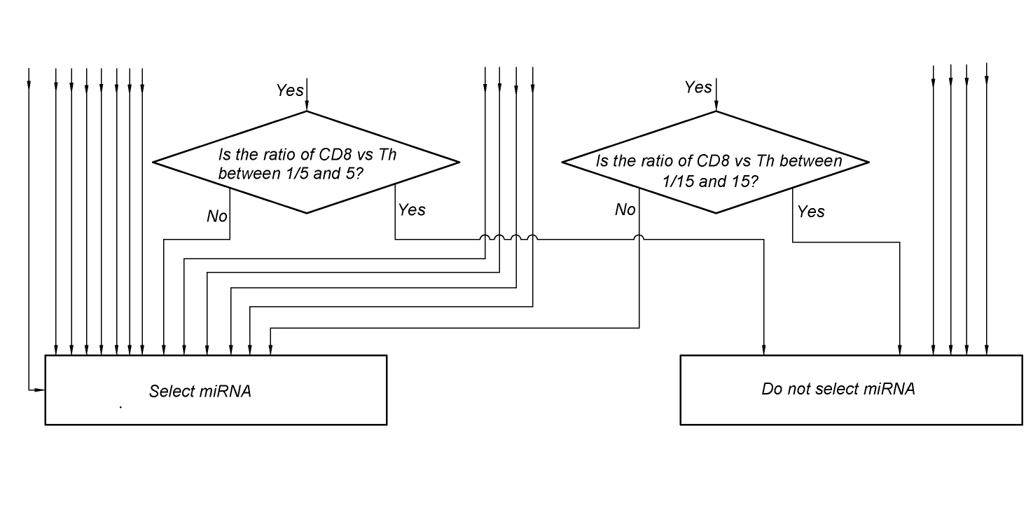

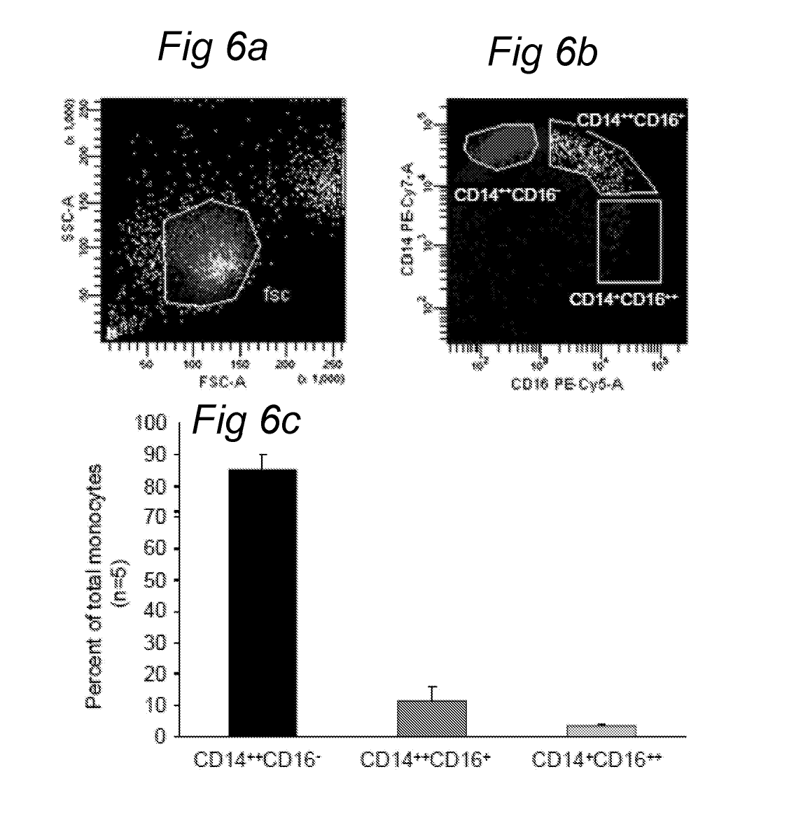

For the selection of miRNAs that reflect phenotypic changes in circulating CD14 cells in patients versus control subjects, miRNAs data sets were generated from a variety of myeloid cells (FIG. 3) using two different technologies. First, to identify all known, novel and candidate expressed miRNAs, we deep-sequenced these different cell types and generated scaled expression data sets as described above. From these we selected 341 miRNAs that were expressed at a level of 100 reads or more in at least one cell type. MiRNAs that fulfilled these criteria were plotted in normalized bargraphs that allowed direct comparison of their expression in all analysed cell types.

One of the potential uses of this myeloid miRNA dataset is their use as biomarkers for altered myeloid phenotypes, and the current “state of the art” medium-high throughput assay for miRNA profiling from cells isolated from tissues are based on quantitative rtPCR by platforms such as TaqMan® Array MicroRNA Cards. Therefore we also generated megaplex miRNA datasets from the myeloid subsets and CD14⁺ cells from 6 angina pectoris patients and control subjects as described above. From the normalized megaplex miRNA data set we selected 282 miRNAs that were expressed in at least one of the myeloid cell types or CD14⁺ cells of patients or controls with a CT value <37 and the expression levels were plotted in bargraphs for selection. Subsequently, we selected 131 miRNA that were differentially expressed in a myeloid phenotype- or patient-specific fashion.

For the design of a custom myeloid megaplex card to establish proof of principle in a patient study, 92 phenotype selective miRNAs were selected by excluding 11 candidate miRNAs and 28 miRNAs that are expressed at CT levels >37 in circulating CD14⁺ cells.

FIG. 4. Random clustering on the basis of myeloid phenotype specific miRNAs distinguishes patients from age and gender matched control subjects

Pilot experiment demonstrating that when microRNAs from isolated CD14 cells are profiled for a selected set of 88 microRNAs the profiles of control subjects and patients are clustered together suggesting a disease related microRNA signature.

FIG. 5. Selection of microRNAs for profiling of CD4⁺ and CD8⁺ cells

For the selection of miRNAs that reflect phenotypic changes in circulating CD4⁺ and CD8⁺ cells in patients versus control subjects, miRNAs data sets were generated from a variety of T-cells using two different technologies. First, to identify all known, novel and candidate expressed miRNAs, we deep sequenced these different cell types and generated scaled expression data sets as described above. A combined miRNA data set was obtained from 6 pooled CD4 positive and CD8 positive T-Cell samples from patients and from a control group and in vitro cultures T helper-1 and T helper-2 differentiated T-Cells. In addition we performed quantitative rtPCR by TaqMan® Array MicroRNA Cards from CD4 positive samples from patients and a control group and four CD8 positive samples from a control group as described above. In addition we obtained megaplex data sets from in vitro generated T helper 1 and T helper 2 samples. Again, to select a panel of T-cell phenotype selective miRNAs we used criteria involving the phenotype specific expression, differential expression in patients and sufficient expression when detected using rtPCR based assays using freshly isolated CD4⁺ and CD8⁺ cells from human blood samples. The flow diagram shows the decision strategy used for the selection protocol.

FIG. 6. FACS analysis and isolation of monocyte subset preparations. A: Representative histogram of a total preparation of CD14⁺ monocytes after negative selection. B: Histogram plotting the expression of CD14 vs. CD16 displaying the three major monocyte subsets being classical monocytes (CD14⁺⁺/CD16⁻), intermediate monocytes (CD14⁺⁺/CD16⁺) and nonclassical monocytes (CD14⁺/CD16⁺⁺). Depicted gates were used for sorting. C: bargraph showing the relative contribution of the three major monocyte subsets to the total number of CD14⁺ monocytes.

### EXAMPLES

### Example 1

### Phenotype-Selective microRNA Profiles from Circulating CD14⁺ Cells to Assess Susceptibility and Progression of Inflammation

Monocytes (CD14⁺) and T-cells (CD4⁺ and CD8⁺)

Peripheral blood mononuclear cells (PBMCs) from 60 ml whole blood were isolated by density gradient centrifugation. To that end, blood was collected in EDTA tubes and diluted with PBS to a final volume of 80 ml. The 80 ml of the diluted EDTA-anticoagulated blood was divided over four Leucosep tubes containing 15 ml Ficoll-paque Plus (Amersham, GE Healthcare Europe) and centrifugated at room temperature in a swinging bucket rotor for 20 minutes at 1000*g with switch-off brakes. The lymphocyte/PBMC rich interphases were pooled and a 50 ul aliquot was used to perform a cell count with a Coulter-counter. Next, the cell suspension was centrifuged at 720*g for 10 minutes at 4 degrees and subsequently the cell pellet was dissolved in 5 ml of 1×BD Imag Cell Separation Buffer (BD Biosciences, USA). After another centrifugation step for 10 minutes at 720*g, cells were incubated with anti-human CD14, CD4 or CD8 magnetic particles (BD Biosciences, USA) for 60 minutes on ice (50 ul magnetic particles per 10⁷ PBMCs). After incubation with the magnetic particles the cell/beads suspension were placed on a pre-cooled BD IMagnet (BD Biosciences, USA) for 10 minutes. With the tube still on the IMagnet the unbound fraction was aspirated. Subsequently the tube with the cell-bead fraction was taken out of the IMagnet and 1 ml of selectionbuffer (BD Biosciences, USA) was added to cells for washing. Again the tube was placed on the IMagnet and unbound fraction was aspirated. Finally the isolated cell fractions (CD14, CD4 or CD8) were resuspended in 20 ul of selectionbuffer and subsequently 500 ul Trizol (Invitrogen, USA) was added to each of the tubes for future RNA isolation. Trizol samples were stored at −80 degrees.

Dendritic Cells (Type-1; CD1a⁺, CD14⁻, CD83) and Dexamethason Treated DCs (Type-2; CD1a⁻, CD14⁺, CD163⁺)

PBMCs and subsequently CD14⁺ cells were isolated as described above. CD14⁺ cell suspension of 0.75*10⁶/ml were plated out in a 6-wells culture plates (Costar, USA). To generate immature type-1 dendritic cells, isolated cells were cultured for 6 days in 2 ml RPMI supplemented with 10 ng/ml IL-4 (Invitrogen, USA), 5 ng/ml GM-CSF (Invitrogen, USA), 10% foetal calf serum (Invitrogen USA), Penicillin 100 U/ml Streptomycin 100 ug/ml (Invitrogen USA) (Hansson G K et al, 2005) Medium with cytokines was refreshed every two days. Phenotype of DCs was determined by FACS analyses, looking at the expression of specific markers CD1a⁺, CD14⁻ and CD83⁻. After 6 days cells were harvested, lysed in 500 μl Trizol, and stored at −80° C. Dex-DCs were generated in a similar fashion, only at the start of differentiation from CD14⁺ cells 400 ng/ml dexamethason was added to the culture medium. The phenotype of the Dex-DCs was determined by FACS analyses looking at CD163 expression and IL-6 production secreted in the culture medium. After 6 days cells were harvested, lysed in 500 μl Trizol, and stored at −80° C.

Type 1 Macrophage (M1) and Type 2 Macrophages (M2)

PBMCs and subsequently CD14⁺ cells were isolated as described above. A CD14⁺ cell suspension of 0.75*10⁶/ml were seeded in a 6-wells culture plate (Costar, USA). M1 macrophages were generated by culturing the CD14⁺ cells for 6 days in 2 ml RPMI supplemented with 5 ng/ml G-MSF (Invitrogen, USA), 10% foetal calfs serum (Invitrogen USA), Penicillin 100 U/ml Streptomycin 100 ug/ml (Invitrogen USA). M2 macrophages were generated by culturing the CD14⁺ cells for 6 days in 2 ml RPMI supplemented with 5 ng/ml M-CSF, 10% foetal calf serum, Penicillin 100 U/ml Streptomycin 100 ug/ml.

Medium with cytokines was refreshed every two days. Phenotype of macrophages was determined by FACS analyses with M1 macrophages: CD14⁺, Mannose receptor⁺ and CD11b⁺⁺ and M2: CD14⁺⁺, Mannose receptor⁻ and CD11b⁺ (Swirski F K et al, 2007). After 7 days cells were harvested, lysed in 500 μl Trizol, and stored at −80° C.

Myeloid Angiogenic Accessory Cells (mEPCs)

PBMCs were isolated as described earlier and plated at a density of 1×10′ cells per cm² on six-well culture plates (Costar) coated with 2% gelatin (Sigma) in M199 medium supplemented with 20% FBS (Invitrogen, USA), 0.05 mg/ml Bovine Pituitary Extract (Invitrogen, USA), Penicillin 100 U/ml Streptomycin 100 ug/ml, and 10 units/nil heparin (Leo Pharma BV, Breda, the Netherlands). Cells were cultured for 7 days as described in Braunersreuther V et al, 2007.

T Cell Supernatant Stimulated Monocytes (T0 h and T48 h)

Human CD14+ monocytes or CD4⁺ T cell-enriched fresh apheresis mononuclear cell preparations from healthy donors were used for the isolation of T-cells and monocytes. T-cell activation was performed as described previously (see Ylitalo R et al, 1994). Total CD14⁺ monocytes (three donors) (T0 h) were cultured for 48 h in fibronectin-coated plates (BD Biosciences) at a density of 1*10⁶ cells/ml in 4:1 mixture of Endocult™ medium (Stem Cell technologies) and pooled T-cell-conditioned medium from 3-4 individual donors. After 48 h the stimulated monocytes (T48 h) were harvested and lysed in 500 ul of Trizol for RNA isolation. The non-stimulated CD14⁺ monocytes serve as a control.

Th1 and Th2 Cells

Isolation of CD4⁺ cells was done as described above. For activation of the T cells, a 96-wells plate was coated with functional grade purified anti-CD3 (aCD3) (eBioscience). A concentration of 2 μg/ml was used. After the anti-CD3 solution was added, the plate was incubated for 5 h at RT. Subsequently the plate was washed twice with PBS. For activation of the T cells, 0.1 μg/ml functional grade purified anti-CD28 (aCD28) (eBioscience) was used. For Th1 polarizing conditions; IL-2 (25 ng/ml), IL-12 (20 ng/ml) and anti-IL4 were added. For Th2 polarizing conditions; IL-2 (25 ng/ml), IL-4 (20 ng/ml) and anti-IFN-γ (5 μg/ml) were added.

The cells were resuspended in 2 ml RPMI supplemented with 10% foetal calfs serum (Invitrogen USA), Penicillin 100 U/ml Streptomycin 100 ug/ml (Invitrogen USA) to a concentration of 5*10⁶ cells/ml, and the cells were grown for 2 days under standard conditions. Subsequently the cells were again resuspended to 5*10⁶ cells/ml, and incubated for 3 additional days under standard conditions. The cells were collected a fraction was used for FACS analysis and stained for the specific markers Tbet-Alexa fluor 647-APC (eBioscience), IL-4-PECy7 (eBioscience) and IFN-y-Percp. Cy5 (eBioscience) to confirm their Th1 or Th2 phenotype. FACS analysis was performed on the BD FACSCanto™ II. The remaining cells were harvested and lysed in 500 ul of Trizol for RNA isolation and stored at −80° C.

Total RNA Isolation

Total RNA was extracted using Trizol reagent (Invitrogen, Carlsbad, USA) according to the manufacturer's instructions with minor modifications. 1.5 μl (20 mg/ml), Glycogen (Roche, USA) was added per sample during the isolation procedure to visualize RNA pellet and precipitation of RNA with isopropanol (Merck, Germany) was done at −20° C. for 30 minutes. The quantity and purity of the isolated RNA were measured with a NanoDrop ND-1000 spectrophotometer (Nanodrop Technologies, Wilmington, Del.). Quality of the RNA was determined on the Bioanalyzer 2100 using an Agilent RNA 6000 Nano chip (Agilent Technologies, USA) (Table 1 and 2).

TABLE 1
RNA quantity and integrity myeloid samples

| | | RIN (RNA INTEGRITY |
|---|---|---|
| sample | ng/ul | NUMBER) |
| used for megaplex only | | |
| CD14 P1 | 189 | 2.5 |
| CD14 P2 | 125 | 1.4 |
| CD14 P3 | 173 | 2.4 |
| CD14 P4 | 35 | 1.1 |
| CD14 P5 | 100 | ND |
| CD14 P6 | 237 | 2.7 |
| C1 CD14 | 267 | 2.4 |
| C2 CD14 | 358 | 9.5 |
| C3 CD14 | 220 | 8.9 |
| C4 CD14 | 403 | 7.9 |
| C5 CD14 | 429 | 8.4 |
| C6 CD14 | 271 | 8.5 |
| used for megaplex and solid deep | | |
| sequencing | | |
| CD14 pool | 674 | ND |
| T48h | 138 | ND |
| T0h | 92 | ND |
| MQ1 pool | 852 | ND |
| MQ2pool | 637 | ND |
| Dcpool | 564 | ND |
| dexDCpool | 867 | ND |
| used for solid deep sequencing only | | |
| CD14C | 306 | 6.2 |
| CD14P | 161 | 2.3 |

ND: not determined

TABLE 2
RNA quantity and integrity Tcell samples used for megaplex and solid
deep sequencing

| | | RIN (RNA INTEGRITY |
|---|---|---|
| sample | ng/ul | NUMBER) |
| Th1 | 400 | 9.2 |
| Th2 | 400 | 9.0 |
| CD4C | 448 | 2.8 |
| CD4P | 210 | ND |
| CD8C | 234 | 7.8 |
| CD8P | 96 | 7.9 |

ND: not determined

Library Construction for Massively Parallel Sequencing

The RNA samples were analyzed in depth for their small RNA content on the SOLiD deep-sequencing platform (Applied Biosystems, Foster City, Calif., USA). In brief, the small RNA fraction from the different cell types was isolated by running the total RNA on a 15% denaturing acrylamide gel, and excising the fraction between 18 nt and 26 nt. For the following steps, the reagents from the SOliD total RNA-seq kit (Applied Biosystems) were used. First a synthetic adaptor was ligated on both sites of the small RNA molecules, followed by first strand cDNA synthesis. The cDNA was subsequently PCR-amplified with adaptor-specific custom-made primers. Each library was amplified with its own set of primers, that contained a specific tag (a short stretch of oligonucleotides) to facilitate sorting of the different samples after deep-sequencing. The final generated deep-sequencing libraries were analyzed by massively parallel sequencing on the SOLiD system (Applied Biosystems).

Computational Analysis of Cloned Small RNA Sequencing Reads

The computational analysis was performed as described by Berezikov et al., 2006. After barcode splitting, masking of adapter sequences and collapsing of identical reads, inserts of length 18 bases and longer were mapped to human genome (GRCh37 assembly) using megablast software (ftp://ftp.ncbi.nlm.nih.gov/blast/). Not all inserts matched perfectly to a genome, and detailed analysis of non-matching sequences indicated that many of them represent known microRNAs with several additional nucleotides added to one of the ends. These non-genomic sequences may be artifacts of the cloning procedure or a result of non-templated modification of mature microRNAs (Allen J B et al, 1991). Such sequences were corrected according to the best blast hit to a genome. Next, for every genomic locus matching to an insert, repeat and gene annotations were retrieved from the Ensembl database (http://www.ensembl.org) and repetitive and gene-coding regions were discarded from further analysis. Genomic regions containing inserts with 100 nt flanks were retrieved from Ensembl and a sliding window of 100 nt was used to calculate RNA secondary structures by RNAfold (Rivier A et al, 1995). Only regions that folded into hairpins and contained an insert in one of the hairpin arms, were used in further analysis. Since every non-redundant insert produced independent hits at this stage, hairpins with overlapping genomic coordinates were merged into one region, tracing locations of matching inserts. In cases when several inserts overlapped, the complete region covered by overlapping inserts was used in downstream calculations as a mature sequence. Next, randfold values were calculated for every sequence in an alignment using mononucleotide shuffling and 1000 iterations (Nockher W A et al, 1998). In addition, for the identified hairpins a number of parameters were calculated, including abundance and 5′ variability of the reads mapped to the hairpin as well as their position relative to the stem and the loop, number of unpaired bases, size of the bulges and Drosha/Dicer overhangs, and number of antisense reads. Based on combinations of these parameters, hairpins were assigned to various confidence levels and then subjected to manual inspection and curation for assignment as confident novel miRNAs or candidate miRNA loci. For comparison of miRNA levels between samples, relative miRNA abundances calculated as the fraction of the total miRNA reads in a given sample were used.

Profiling microRNAs by TaqMan® Array MicroRNA Cards

For miRNA cDNA synthesis, 350 ng of total RNA was reverse transcribed using the miRNA reverse transcription kit (Applied Biosystems) in combination with the stem-loop Megaplex Human primer pools A V2.1 (Applied Biosystems) according to manufacturer's instructions.

For each cDNA sample, 384 microRNAs including 6 controls (RNU44, RNU48, 4*U6), were profiled using TaqMan® Array MicroRNA Human Card A V2.0 (Applied Biosystems) according to manufacturer's instructions. All arrays were run on a 7900HT Fast Real-Time PCR System (Applied Biosystems) and default thermal-cycling conditions. For each array the obtained Ct-values were converted to relative quantities normalized to RNU48.

Selection of Phenotype Selective miRNAs from Circulating CD14+ and CD4⁺/CD8⁺ Cells

Selection of Myeloid microRNAs for Profiling of CD14⁺ Cells

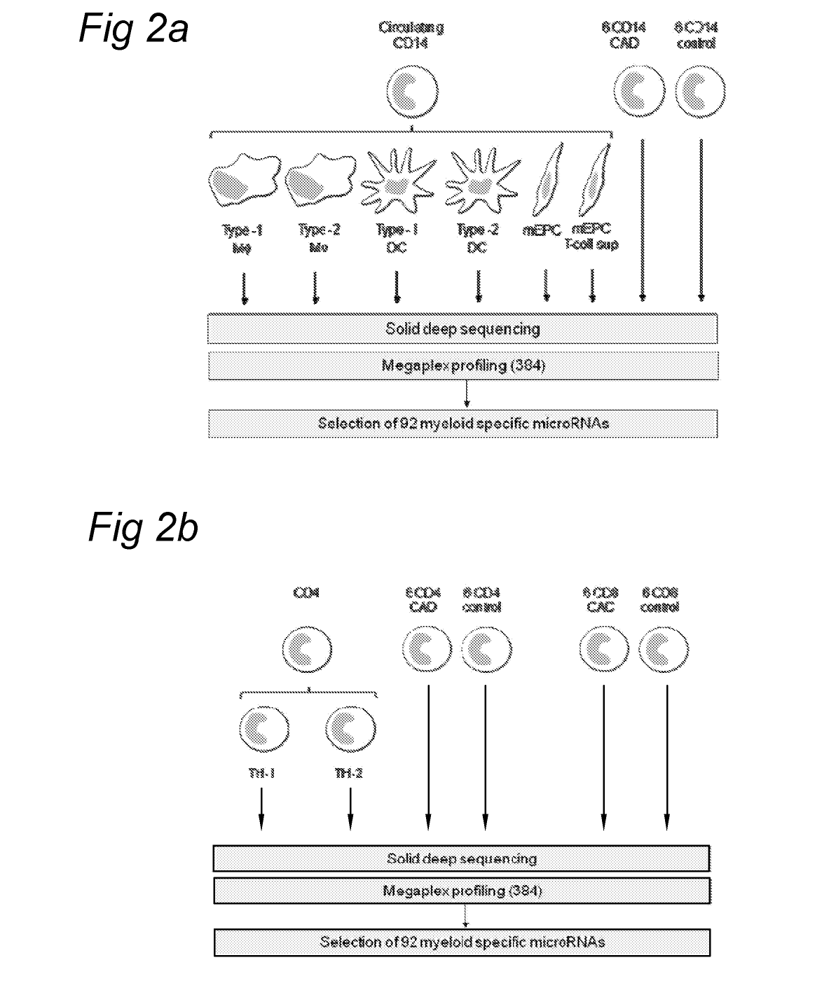

For the selection of miRNAs that reflect phenotypic changes in circulating CD14 cells in patients versus control subjects, miRNAs data sets were generated from a variety of myeloid cells (FIG. 2A) using two different technologies. First, to identify all known, novel and candidate expressed miRNAs, we deep-sequenced these different cell types and generated scaled expression data sets as described above. From these we selected 341 miRNAs that were expressed at a level of 100 reads or more in at least one cell type. MiRNAs that fulfilled these criteria were plotted in normalized bargraphs to allow for a direct comparison of their expression in all analyzed cell types.

One of the potential uses of this myeloid miRNA dataset is their use as biomarkers for altered myeloid phenotypes, and the current “state of the art” medium-high throughput assay for miRNA profiling from cells isolated from tissues are based on quantitative rtPCR by platforms such as TaqMan® Array MicroRNA Cards. Therefore we also generated megaplex miRNA datasets from the myeloid subsets and CD14⁺ cells from 6 angina pectoris patients and control subjects as described above. From the normalized megaplex miRNA data set we selected 282 miRNAs that were expressed in at least one of the myeloid cell types or CD14⁺ cells of patients or controls with a CT value <37 and the expression levels were plotted in paragraphs for selection.

Subsequently, we selected miRNA that were differentially expressed in a myeloid phenotype- or patient-specific fashion (FIG. 3). To that end, we first selected 131 miRNAs that were differentially expressed for at least 1.5-fold in myeloid-specific or patient vs. control subsets in both the megaplex and/or deep sequence-data sets. For the design of a custom myeloid megaplex card to establish proof of principle in a patient study, 92 phenotype selective miRNAs were selected by excluding 11 candidate miRNAs and 28 miRNAs that are expressed at CT levels >37 in circulating CD14⁺ cells (table 3).

To validate the concept of the use of CD14 cell microRNAs as biomarkers for CVD we profiled the microRNAs of isolated CD14 cells from 6 patients and 6 controls using commercially 384 megaplex plates. When the data are subjected to un-biased cluster analysis and we select for 88 microRNAs (preliminary selection that is now extended to 92) that were shown to be associated with phenotypic alterations in myeloid cells, the patients and the control profiles cluster together in patients versus controls (FIG. 4).

TABLE 3
Phenotypes elective miRNAs for
circulating CD14⁺ cells

| Seq | | |
|---|---|---|
| ID | miRNA | Target sequence |
| 1 | hsa-miR-1 | UGGAAUGUAAAGAAGUAUGUAU |
| 2 | hsa-let-7f | UGAGGUAGUAGAUUGUAUAGUU |
| 3 | hsa-miR-10a | UACCCUGUAGAUCCGAAUUUGUG |
| 4 | hsa-miR-15b | UAGCAGCACAUCAUGGUUUACA |
| 5 | hsa-miR-18b | UAAGGUGCAUCUAGUGCAGUUAG |
| 6 | hsa-miR-26a | UUCAAGUAAUCCAGGAUAGGCU |
| 7 | hsa-miR-27b | UUCACAGUGGCUAAGUUCUGC |
| 8 | hsa-miR-28-3p | CACUAGAUUGUGAGCUCCUGGA |
| 9 | hsa-miR-28-5p | AAGGAGCUCACAGUCUAUUGAG |
| 10 | hsa-miR-32 | UAUUGCACAUUACUAAGUUGCA |
| 11 | hsa-miR-92a | UAUUGCACUUGUCCCGGCCUGU |
| 12 | hsa-miR-98 | UGAGGUAGUAAGUUGUAUUGUU |
| 13 | hsa-miR-99a | AACCCGUAGAUCCGAUCUUGUG |
| 14 | hsa-miR-99b | CACCCGUAGAACCGACCUUGCG |
| 15 | hsa-miR-100 | AACCCGUAGAUCCGAACUUGUG |
| 16 | hsa-miR-125b | UCCCUGAGACCCUAACUUGUGA |
| 17 | hsa-miR-128 | UCACAGUGAACCGGUCUCUUU |
| 18 | hsa-miR-130a | CAGUGCAAUGUUAAAAGGGCAU |
| 19 | hsa-miR-130b | CAGUGCAAUGAUGAAAGGGCAU |
| 20 | hsa-miR-133a | UUUGGUCCCCUUCAACCAGCUG |
| 21 | hsa-miR-133b | UUUGGUCCCCUUCAACCAGCUA |
| 22 | hsa-miR-138 | AGCUGGUGUUGUGAAUCAGGCCG |
| 23 | hsa-miR-140-5p | CAGUGGUUUUACCCUAUGGUAG |
| 24 | hsa-miR-142-5p | CAUAAAGUAGAAAGCACUACU |
| 25 | hsa-miR-145 | GUCCAGUUUUCCCAGGAAUCCCU |
| 26 | hsa-miR-193a-5p | UGGGUCUUUGCGGGCGAGAUGA |
| 27 | hsa-miR-200a | UAACACUGUCUGGUAACGAUGU |
| 28 | hsa-miR-218 | UUGUGCUUGAUCUAACCAUGU |
| 29 | hsa-miR-342-3p | UCUCACACAGAAAUCGCACCCGU |
| 30 | hsa-miR-365 | UAAUGCCCCUAAAAAUCCUUAU |
| 31 | hsa-miR-422a | ACUGGACUUAGGGUCAGAAGGC |
| 32 | hsa-miR-424 | CAGCAGCAAUUCAUGUUUUGAA |
| 33 | hsa-miR-449a | UGGCAGUGUAUUGUUAGCUGGU |
| 34 | hsa-miR-449b | AGGCAGUGUAUUGUUAGCUGGC |
| 35 | hsa-miR-450b-5p | UUUUGCAAUAUGUUCCUGAAUA |
| 36 | hsa-miR-451 | AAACCGUUACCAUUACUGAGUU |
| 37 | hsa-miR-487b | AAUCGUACAGGGUCAUCCACUU |
| 38 | hsa-miR-491-5p | AGUGGGGAACCCUUCCAUGAGG |
| 39 | hsa-miR-500 | UAAUCCUUGCUACCUGGGUGAGA |
| 40 | hsa-miR-501-5p | AAUCCUUUGUCCCUGGGUGAGA |
| 41 | hsa-miR-503 | UAGCAGCGGGAACAGUUCUGCAG |
| 42 | hsa-miR-574-3p | CACGCUCAUGCACACACCCACA |
| 43 | hsa-miR-590-5p | GAGCUUAUUCAUAAAAGUGCAG |
| 44 | hsa-miR-628-5p | AUGCUGACAUAUUUACUAGAGG |
| 45 | hsa-miR-708 | AAGGAGCUUACAAUCUAGCUGGG |
| 46 | hsa-miR-885-5p | UCCAUUACACUACCCUGCCUCU |
| 47 | hsa-miR-376c | AACAUAGAGGAAAUUCCACGU |
| 48 | hsa-mir-124 | UAAGGCACGCGGUGAAUGCC |
| 49 | hsa-let-7d | AGAGGUAGUAGGUUGCAUAGUU |
| 50 | hsa-let-7e | UGAGGUAGGAGGUUGUAUAGUU |
| 51 | hsa-let-7g | UGAGGUAGUAGUUUGUACAGUU |
| 52 | hsa-miR-17 | CAAAGUGCUUACAGUGCAGGUAG |
| 53 | hsa-miR-19a | UGUGCAAAUCUAUGCAAAACUGA |
| 54 | hsa-miR-19b | UGUGCAAAUCCAUGCAAAACUGA |
| 55 | hsa-miR-20a | UAAAGUGCUUAUAGUGCAGGUAG |
| 56 | hsa-miR-20b | CAAAGUGCUCAUAGUGCAGGUAG |
| 57 | hsa-miR-21 | UAGCUUAUCAGACUGAUGUUGA |
| 58 | hsa-miR-26b | UUCAAGUAAUUCAGGAUAGGU |
| 59 | hsa-miR-27a | UUCACAGUGGCUAAGUUCCGC |
| 60 | hsa-miR-29a | UAGCACCAUCUGAAAUCGGUUA |
| 61 | hsa-miR-31 | AGGCAAGAUGCUGGCAUAGCU |
| 62 | hsa-miR-34a | UGGCAGUGUCUUAGCUGGUUGU |
| 63 | hsa-miR-93 | CAAAGUGCUGUUCGUGCAGGUAG |
| 64 | hsa-miR-103 | AGCAGCAUUGUACAGGGCUAUGA |
| 65 | hsa-miR-106b | UAAAGUGCUGACAGUGCAGAU |
| 66 | hsa-miR-125a-5p | UCCCUGAGACCCUUUAACCUGUG |
| 67 | hsa-miR-126 | UCGUACCGUGAGUAAUAAUGCG |
| 68 | hsa-miR-132 | UAACAGUCUACAGCCAUGGUCG |
| 69 | hsa-miR-139-5p | UCUACAGUGCACGUGUCUCCAG |
| 70 | hsa-miR-142-3p | UGUAGUGUUUCCUACUUUAUGGA |
| 71 | hsa-miR-146a | UGAGAACUGAAUUCCAUGGGUU |
| 72 | hsa-miR-146b-5p | UGAGAACUGAAUUCCAUAGGCU |
| 73 | hsa-miR-150 | UCUCCCAACCCUUGUACCAGUG |
| 74 | hsa-miR-181a | AACAUUCAACGCUGUCGGUGAGU |
| 75 | hsa-miR-191 | CAACGGAAUCCCAAAAGCAGCUG |
| 76 | hsa-miR-193b | AACUGGCCCUCAAAGUCCCGCU |
| 77 | hsa-miR-195 | UAGCAGCACAGAAAUAUUGGC |
| 78 | hsa-miR-197 | UUCACCACCUUCUCCACCCAGC |
| 79 | hsa-miR-210 | CUGUGCGUGUGACAGCGGCUGA |
| 80 | hsa-miR-221 | AGCUACAUUGUCUGCUGGGUUUC |
| 81 | hsa-miR-223 | UGUCAGUUUGUCAAAUACCCCA |
| 82 | hsa-miR-328 | CUGGCCCUCUCUGCCCUUCCGU |
| 83 | hsa-miR-331-3p | GCCCCUGGGCCUAUCCUAGAA |
| 84 | hsa-miR-155 | UUAAUGCUAAUCGUGAUAGGGGU |
| 85 | hsa-let-7b | UGAGGUAGUAGGUUGUGUGGUU |
| 86 | hsa-miR-374b | AUAUAAUACAACCUGCUAAGUG |
| 87 | hsa-miR-423-5p | UGAGGGGCAGAGAGCGAGACUUU |
| 88 | hsa-miR-486-3p | CGGGGCAGCUCAGUACAGGAU |
| 89 | hsa-miR-671-3p | UCCGGUUCUCAGGGCUCCACC |
| 90 | hsa-miR-212 | UAACAGUCUCCAGUCACGGCC |
| 91 | hsa-miR-345 | GCUGACUCCUAGUCCAGGGCUC |
| 92 | hsa-let-7i | UGAGGUAGUAGUUUGUGCUGU |

### Example 2

### Phenotype-Selective microRNA Profiles from Circulating CD4⁺ and CD8⁺ Cells to Assess Susceptibility and Progression of Inflammation

For the selection of miRNAs that reflect phenotypic changes in circulating CD4⁺ and CD8⁺ cells in patients versus control subjects, miRNAs data sets were generated from a variety of T-cells using two different technologies. First, to identify all known, novel and candidate expressed miRNAs, we deep-sequenced these different cell types (FIG. 2B) and generated scaled expression data sets as described above. A combined miRNA data set was obtained from 6 pooled CD4 positive and CD8 positive T-Cell samples from patients and from a control group and in vitro cultures T helper-1 and T helper-2 differentiated T-Cells. In addition we performed quantitative rtPCR by TaqMan® Array MicroRNA Cards from CD4 positive samples from patients and a control group and four CD8 positive samples from a control group as described above. In addition we obtained megaplex data sets from in vitro generated T helper 1 and T helper 2 samples. Again, to select a panel of T-cell phenotype selective miRNAs (table 4) we used criteria involving the phenotype specific expression, differential expression in patients and sufficient expression when detected using rtPCR based assays using freshly isolated CD4⁺ and CD8⁺ cells from human blood samples. The flow diagram in FIG. 5 shows the decision strategy used for the selection protocol.

TABLE 4
Phenotype selective miRNAs for
circulating CD4⁺ and CD8⁺ cells

| Seq | | |
|---|---|---|
| ID | miRNA | Target sequence |
| 49 | hsa-let-7d | AGAGGUAGUAGGUUGCAUAGUU |
| 50 | hsa-let-7e | UGAGGUAGGAGGUUGUAUAGUU |
| 51 | hsa-let-7g | UGAGGUAGUAGUUUGUACAGUU |
| 52 | hsa-miR-17 | CAAAGUGCUUACAGUGCAGGUAG |
| 53 | hsa-miR-19a | UGUGCAAAUCUAUGCAAAACUGA |
| 54 | hsa-miR-19b | UGUGCAAAUCCAUGCAAAACUGA |
| 55 | hsa-miR-20a | UAAAGUGCUUAUAGUGCAGGUAG |
| 56 | hsa-miR-20b | CAAAGUGCUCAUAGUGCAGGUAG |
| 57 | hsa-miR-21 | UAGCUUAUCAGACUGAUGUUGA |
| 58 | hsa-miR-26b | UUCAAGUAAUUCAGGAUAGGU |
| 59 | hsa-miR-27a | UUCACAGUGGCUAAGUUCCGC |
| 60 | hsa-miR-29a | UAGCACCAUCUGAAAUCGGUUA |
| 61 | hsa-miR-31 | AGGCAAGAUGCUGGCAUAGCU |
| 62 | hsa-miR-34a | UGGCAGUGUCUUAGCUGGUUGU |
| 63 | hsa-miR-93 | CAAAGUGCUGUUCGUGCAGGUAG |
| 64 | hsa-miR-103 | AGCAGCAUUGUACAGGGCUAUGA |
| 65 | hsa-miR-106b | UAAAGUGCUGACAGUGCAGAU |
| 66 | hsa-miR-125a-5p | UCCCUGAGACCCUUUAACCUGUG |
| 67 | hsa-miR-126 | UCGUACCGUGAGUAAUAAUGCG |
| 68 | hsa-miR-132 | UAACAGUCUACAGCCAUGGUCG |
| 69 | hsa-miR-139-5p | UCUACAGUGCACGUGUCUCCAG |
| 70 | hsa-miR-142-3p | UGUAGUGUUUCCUACUUUAUGGA |
| 71 | hsa-miR-146a | UGAGAACUGAAUUCCAUGGGUU |
| 72 | hsa-miR-146b-5p | UGAGAACUGAAUUCCAUAGGCU |
| 73 | hsa-miR-150 | UCUCCCAACCCUUGUACCAGUG |
| 74 | hsa-miR-181a | AACAUUCAACGCUGUCGGUGAGU |
| 75 | hsa-miR-191 | CAACGGAAUCCCAAAAGCAGCUG |
| 76 | hsa-miR-193b | AACUGGCCCUCAAAGUCCCGCU |
| 77 | hsa-miR-195 | UAGCAGCACAGAAAUAUUGGC |
| 78 | hsa-miR-197 | UUCACCACCUUCUCCACCCAGC |
| 79 | hsa-miR-210 | CUGUGCGUGUGACAGCGGCUGA |
| 80 | hsa-miR-221 | AGCUACAUUGUCUGCUGGGUUUC |
| 81 | hsa-miR-223 | UGUCAGUUUGUCAAAUACCCCA |
| 82 | hsa-miR-328 | CUGGCCCUCUCUGCCCUUCCGU |
| 83 | hsa-miR-331-3p | GCCCCUGGGCCUAUCCUAGAA |
| 84 | hsa-miR-155 | UUAAUGCUAAUCGUGAUAGGGGU |
| 85 | hsa-let-7b | UGAGGUAGUAGGUUGUGUGGUU |
| 86 | hsa-miR-374b | AUAUAAUACAACCUGCUAAGUG |
| 87 | hsa-miR-423-5p | UGAGGGGCAGAGAGCGAGACUUU |
| 88 | hsa-miR-486-3p | CGGGGCAGCUCAGUACAGGAU |
| 89 | hsa-miR-671-3p | UCCGGUUCUCAGGGCUCCACC |
| 90 | hsa-miR-212 | UAACAGUCUCCAGUCACGGCC |
| 91 | hsa-miR-345 | GCUGACUCCUAGUCCAGGGCUC |
| 92 | hsa-let-7i | UGAGGUAGUAGUUUGUGCUGU |
| 93 | hsa-let-7a | UGAGGUAGUAGGUUGUAUAGUU |
| 94 | hsa-miR-15a | UAGCAGCACAUAAUGGUUUGUG |
| 95 | hsa-miR-18a | UAAGGUGCAUCUAGUGCAGAUAG |
| 96 | hsa-miR-24 | UGGCUCAGUUCAGCAGGAACAG |
| 97 | hsa-miR-29c | UAGCACCAUUUGAAAUCGGUUA |
| 98 | hsa-miR-30b | UGUAAACAUCCUACACUCAGCU |
| 99 | hsa-miR-34c-5p | AGGCAGUGUAGUUAGCUGAUUGC |
| 100 | hsa-miR-106a | AAAAGUGCUUACAGUGCAGGUAG |
| 101 | hsa-miR-135b | UAUGGCUUUUCAUUCCUAUGUGA |
| 102 | hsa-miR-185 | UGGAGAGAAAGGCAGUUCCUGA |
| 103 | hsa-miR-186 | CAAAGAAUUCUCCUUUUGGGCU |
| 104 | hsa-miR-199a-3p | ACAGUAGUCUGCACAUUGGUUA |
| 105 | hsa-miR-199b-5p | CCCAGUGUUUAGACUAUCUGUUC |
| 106 | hsa-miR-200c | UAAUACUGCCGGGUAAUGAUGGA |
| 107 | hsa-miR-222 | AGCUACAUCUGGCUACUGGGU |
| 108 | hsa-miR-326 | CCUCUGGGCCCUUCCUCCAG |
| 109 | hsa-miR-330-3p | GCAAAGCACACGGCCUGCAGAGA |
| 110 | hsa-miR-335 | UCAAGAGCAAUAACGAAAAAUGU |
| 111 | hsa-miR-337-5p | GAACGGCUUCAUACAGGAGUU |
| 112 | hsa-miR-339-5p | UCCCUGUCCUCCAGGAGCUCACG |
| 113 | hsa-miR-340 | UUAUAAAGCAAUGAGACUGAUU |
| 114 | hsa-miR-374a | UUAUAAUACAACCUGAUAAGUG |
| 115 | hsa-miR-409-5p | AGGUUACCCGAGCAACUUUGCAU |
| 116 | hsa-miR-425 | AAUGACACGAUCACUCCCGUUGA |
| 117 | hsa-miR-454 | UAGUGCAAUAUUGCUUAUAGGGU |
| 118 | hsa-miR-455-5p | UAUGUGCCUUUGGACUACAUCG |
| 119 | hsa-miR-484 | UCAGGCUCAGUCCCCUCCCGAU |
| 120 | hsa-miR-532-5p | CAUGCCUUGAGUGUAGGACCGU |
| 121 | hsa-miR-542-5p | UCGGGGAUCAUCAUGUCACGAGA |
| 122 | hsa-miR-582-5p | UUACAGUUGUUCAACCAGUUACU |
| 123 | hsa-miR-598 | UACGUCAUCGUUGUCAUCGUCA |
| 124 | hsa-miR-625 | AGGGGGAAAGUUCUAUAGUCC |
| 125 | hsa-miR-654-5p | UGGUGGGCCGCAGAACAUGUGC |
| 126 | hsa-miR-660 | UACCCAUUGCAUAUCGGAGUUG |
| 127 | hsa-miR-744 | UGCGGGGCUAGGGCUAACAGCA |
| 128 | hsa-miR-886-5p | CGGGUCGGAGUUAGCUCAAGCGG |
| 129 | hsa-mir-1308 | GCAUGGGUGGUUCAGUGGUAG |
| 130 | hsa-mir-181b | AACAUUCAUUGCUGUCGGUGGGU |
| 131 | hsa-mir-1979 | CUCCCACUGCUUCACUUGACUAGC |
| 132 | hsa-mir-2110 | UUGGGGAAACGGCCGCUGAGUGA |
| 133 | hsa-mir-320a | AAAAGCUGGGUUGAGAGGGCGAAA |
| 134 | hsa-mir-378c | ACUGGACUUGGAGUCAGAAG |
| 135 | hsa-mir-4284 | GCUCACAUCACCCCAUAAACAAA |
| 136 | hsa-mir-196a | UAGGUAGUUUCAUGUUGUUG |
| 137 | hsa-mir-1249 | ACGCCCUUCCCCCCCUUCUUCA |
| 138 | hsa-mir-1271 | CUUGGCACCUAGCAAGCACUCA |
| 139 | hsa-mir-378 | CUCCUGACUCCAGGUCCUGUGU |
| 140 | hsa-mir-942 | UCUUCUCUGUUUUGGCCAUGUG |

TABLE 5
Phenotype selective group a miRNAs
for circulating CD14⁺ cells

| Seq ID | miRNA | Target sequence |
|---|---|---|
| 29 | hsa-miR-342-3p | UCUCACACAGAAAUCGCACCCGU |
| 33 | hsa-miR-449a | UGGCAGUGUAUUGUUAGCUGGU |
| 43 | hsa-miR-590-5p | GAGCUUAUUCAUAAAAGUGCAG |
| 68 | hsa-miR-132 | UAACAGUCUACAGCCAUGGUCG |
| 71 | hsa-miR-146a | UGAGAACUGAAUUCCAUGGGUU |
| 90 | hsa-miR-212 | UAACAGUCUCCAGUCACGGCC |

TABLE 6
Phenotype selective group b miRNAs
for circulating CD14⁺ cells

| Seq ID | miRNA | Target sequence |
|---|---|---|
| 27 | hsa-miR-200a | UAACACUGUCUGGUAACGAUGU |
| 34 | hsa-miR-449b | AGGCAGUGUAUUGUUAGCUGGC |
| 37 | hsa-miR-487b | AAUCGUACAGGGUCAUCCACUU |
| 45 | hsa-miR-708 | AAGGAGCUUACAAUCUAGCUGGG |
| 47 | hsa-miR-376c | AACAUAGAGGAAAUUCCACGU |
| 79 | hsa-miR-210 | CUGUGCGUGUGACAGCGGCUGA |

TABLE 7
Phenotype selective group c miRNAs
for circulating CD14⁺ cells

| Seq ID | miRNA | Target sequence |
|---|---|---|
| 3 | hsa-miR-10a | UACCCUGUAGAUCCGAAUUUGUG |
| 10 | hsa-miR-32 | UAUUGCACAUUACUAAGUUGCA |
| 14 | hsa-miR-99b | CACCCGUAGAACCGACCUUGCG |
| 15 | hsa-miR-100 | AACCCGUAGAUCCGAACUUGUG |
| 17 | hsa-miR-128 | UCACAGUGAACCGGUCUCUUU |
| 18 | hsa-miR-130a | CAGUGCAAUGUUAAAAGGGCAU |
| 19 | hsa-miR-130b | CAGUGCAAUGAUGAAAGGGCAU |
| 20 | hsa-miR-133a | UUUGGUCCCCUUCAACCAGCUG |
| 25 | hsa-miR-145 | GUCCAGUUUUCCCAGGAAUCCCU |
| 26 | hsa-miR-193a-5p | UGGGUCUUUGCGGGCGAGAUGA |
| 30 | hsa-miR-365 | UAAUGCCCCUAAAAAUCCUUAU |
| 39 | hsa-miR-500 | UAAUCCUUGCUACCUGGGUGAGA |
| 42 | hsa-miR-574-3p | CACGCUCAUGCACACACCCACA |
| 44 | hsa-miR-628-5p | AUGCUGACAUAUUUACUAGAGG |
| 46 | hsa-miR-885-5p | UCCAUUACACUACCCUGCCUCU |
| 52 | hsa-miR-17 | CAAAGUGCUUACAGUGCAGGUAG |
| 53 | hsa-miR-19a | UGUGCAAAUCUAUGCAAAACUGA |
| 54 | hsa-miR-19b | UGUGCAAAUCCAUGCAAAACUGA |
| 55 | hsa-miR-20a | UAAAGUGCUUAUAGUGCAGGUAG |
| 56 | hsa-miR-20b | CAAAGUGCUCAUAGUGCAGGUAG |
| 62 | hsa-miR-34a | UGGCAGUGUCUUAGCUGGUUGU |
| 63 | hsa-miR-93 | CAAAGUGCUGUUCGUGCAGGUAG |
| 66 | hsa-miR-125a-5p | UCCCUGAGACCCUUUAACCUGUG |
| 72 | hsa-miR-146b-5p | UGAGAACUGAAUUCCAUAGGCU |
| 75 | hsa-miR-191 | CAACGGAAUCCCAAAAGCAGCUG |
| 80 | hsa-miR-221 | AGCUACAUUGUCUGCUGGGUUUC |
| 81 | hsa-miR-223 | UGUCAGUUUGUCAAAUACCCCA |
| 86 | hsa-miR-374b | AUAUAAUACAACCUGCUAAGUG |
| 88 | hsa-miR-486-3p | CGGGGCAGCUCAGUACAGGAU |
| 91 | hsa-miR-345 | GCUGACUCCUAGUCCAGGGCUC |

### Example 3

### Subsets of Phenotype-Selective microRNA Profiles from Circulating CD14⁺ Cells to Assess Susceptibility and Progression of Inflammation

Selection of Monocyte Subset-Selective miRNAs

To determine miRNA expression profiles of circulating monocyte subsets we isolated total monocyte preparations from peripheral blood by negative selection, and sorted for CD14⁺⁺/CD16⁻, CD14⁺⁺/CD16⁺ and CD14⁺/CD16⁺⁺ monocytes (FIG. 6AB). As shown in FIG. 6C, the majority of monocytes were gated into the CD14⁺⁺/CD16⁻ classical monocyte subpopulation (85.4%±4.7%), while the CD14⁺⁺/CD16⁺ intermediate monocytes accounted for approximately 11% of the total monocytes (11.3%±4.6%). CD14⁺/CD16⁺⁺ nonclassical monocytes represented but 3% of the total gated monocytes (3.3%±0.7%).

Monocyte subsets were sorted from 4 independent donors, fractions were pooled and used for isolation of total RNA. Subsequently, miRNA expression profiles were determined using custom TaqMan® Array MicroRNA Cards harboring the 92 myeloid-phenotype selective miRNA assays described above. Datasets generated were imported into BRB-ArrayTools (Version: 4.1.0-Beta_{—}2 Release http://linus.nci.nih.gov/BRB-ArrayTools.html). Signal intensities were log transformed and normalized using the endogenous control microRNA gene RNU48. The class comparison function integrated in BRB-ArrayTools was used to identify differentially expressed miRNAs among the different monocyte subsets. The obtained results were visualised in a combined heatmap and table that depicts the P values for differential expression of the individual miRNAs (not shown).

Following this analyses we identified three clusters of miRNAs that are differentially expressed in the monocyte subsets. Clusters are nucleated around miRNAs that achieved significance for differential expression between the monocytes subsets within the group of four donors (indicated with ^{#}).

Cluster A comprises 7 miRNAs (miR-218, miR-449a, miR-212, miR-132^{#}, miR-342-3p^{#}, mir-146a^{#}, mir-590-5p) that are predominantly up regulated compared to global CD14⁺ miRNA expression profiles.

Cluster B comprises 6 miRNAs (miR-449b, miR-487b^{#}, miR200a, miR-210, miR708, miR-376c) that are differentially expressed in the monocyte subsets.

Cluster C comprises 32 miRNAs (miR-133a, miR-99a, miR-150, miR-126, miR-10a, miR-34a, miR-32^{#}, miR191^{#}, miR885-5p^{#}, miR125a-5p^{#}, miR99b, miR422a, miR146b-5p, miR-130a, miR142-5p, miR-100, miR-130b, miR-486-3p, miR-500, miR128, miR-145^{#}, miR-221, miR-574-3p, miR-106b^{#}, miR-19a, miR-19b, miR-365^{#}, miR-15b^{#}, miR-155^{#}, miR-345^{#}, miR-20a^{#}, miR-93^{#}, miR-20b^{#}, miR-223^{#}, miR-17^{#}, miR193a-5p, miR374b^{#}, miR-628-5p^{#})

Material and Methods

Monocyte Subset Isolation

Monocytes were isolated from buffy coats by negative selection. Briefly, blood was diluted with Dulbecco's PBS (dPBS), after which Ficoll Paque-PLUS (GE Healthcare Life Sciences) was carefully added to the bottom of the conical. Subsequently, the samples were centrifuged at xg for 20 minutes at room temperature, after which the interphase was removed and washed 4× with dPBS to remove thrombocytes, yielding the peripheral blood mononuclear (PBMC) fraction. All subsequent steps were performed on ice.

Monocytes were isolated from the PBMC fraction using the pan-monocyte isolation kit (Miltenyi) as per manufacturer's instructions. The total number of monocytes isolated was determined by Sysmex analysis (Sysmex 6800), after which 5 million monocytes were harvested and resuspended in Trizol (Invitrogen) for RNA isolation. To the remaining monocytes, CD16-Pc5 (Beckman Coulter) and CD14-Pc7 antibodies were added and incubated for 30 minutes on ice. Unbound antibody was removed by adding 1 mL ice-cold FACS buffer (dPBS containing 1% bovine serum albumin and 0.1% sodium azide). The cells were centrifuged at xg for 3 minutes, after which the cell pellet was resuspended and washed with ice-cold FACS buffer. Following centrifugation, the pellet was resuspended in FACS buffer and sorted for into CD14⁺⁺/CD16⁻, CD14⁺⁺/CD16⁺ and CD14^{dim}/CD16⁺ monocyte subpopulations (FACSAriall, Becton Dickinson, Breda, the Netherlands). Upon completion of FACS sorting, the cells were pelleted and resuspended in Trizol for RNA isolation.

### Example 4

### Validation of the CD14⁺ Phenotype Selective miRNAs

Following the selection of CD14⁺ phenotype selective miRNAs the concept that differential expression of the selected microRNAs associated with cardiovascular risk factors and/or outcome was validated. To that end total RNA was isolated from purified profiled CD14⁺ from 441 patients included in the “CIRCULATING CELLS study” (see example 3). Nine months after inclusion, patients are followed for adverse cardiovascular events and death. Extensive case record forms including medical history, risk factors, medication, extent and severity of coronary artery disease, laboratory measurements, and final procedural result were filled in at inclusion (data not shown).

Patients scheduled for coronary angiography were included in this study. Exclusion criteria were: age <18 years, inability to give informed consent, suspected drug or alcohol abuse, serious concomitant disease, serious recent infectious disease in the last 6 weeks or suspected elevated state of the immune system, and non-cooperativeness and patients with ST-elevation myocardial infarction (STEMI). Most patients underwent coronary angiography or PCI. The anatomical severity of coronary artery disease was assessed by calculating the Syntax Score of each patient. The Syntax Score (SS) was introduced in the Synergy between Percutaneous Coronary Intervention with Taxus and Cardiac Surgery (SYNTAX) study which compared the effectiveness of treatment of PCI and coronary bypass surgery for patients with three-vessel or left main stem coronary artery disease or both. It provides an estimate of the complexity of coronary artery disease.

SYNTAX study compared the effectiveness of treatment of PCI and coronary bypass surgery for patients with three-vessel or left main stem coronary artery disease or both (Serruys P W et al, or Sjanos G et al). It provides an estimate of the complexity of coronary artery disease. The SYNTAX score is an angiographic tool grading the complexity of coronary artery disease (CAD).

From all patients, up to 100 ml blood was collected via the arterial sheath catheter directly after insertion and 65 ml EDTA blood were reserved for cell fractionation and diluted with PBS to a final volume of 80 ml, filled out in 50 ml Leucosep tubes (Greiner Bio-One, Alphen, Netherlands) containing 15 ml Ficoll-paque Plus (GE Healthcare, Diegem, Netherlands) each. After centrifugation for 20 min at 1000 g, the PBMC rich interphase was collected into fresh tubes. ⅓ of the PBMC fraction was resuspended in Imag Cell Separation Buffer (BD Biosciences, Breda, Netherlands) transferred into a vial of 2 ml (for CD14⁺ cell isolation). Cells were incubated with anti-human CD14 magnetic particles (BD Biosciences, Breda, Netherlands) for 60 min on ice. Magnetic selection was performed using pre-cooled magnets kept on ice during the separation. The supernatant was aspirated and discarded and the cells were resuspended in Trizol. Aliquots were frozen and stored at −80° C. for RNA isolation and future transcriptomic analyses (miRNA).

### Data Integration

For data integration from the various assays, a clinical database (CircuCel DB) that provides both clinical parameters for the subjects recruited and serves as knowledge base for subsequent biomarker discovery has been created. The CircuCel DB was designed to store any type of assay (microarrays, flow cytometry, proteomics, etc.) results or summarization outcome of these results and to provide analysis support, which includes experimental design and quality control criteria for subsequent data analysis.

Follow Up and Endpoints

The primary endpoint of this study is the occurrence of major adverse cardiovascular events (MACE) within 9 months after inclusion, defined as: death, myocardial infarction (MI), percutaneous coronary intervention (PCI), coronary artery bypass grafting (CABG), cardiovascular accident (CVA). The secondary endpoint is the occurrence of MACE or peripheral vascular intervention. During follow-up, patients were questioned about the occurrence of cardiovascular events, defined as cardiovascular death, myocardial infarction, repeat revascularization (PCI or CABG), recurrent angina, CVA, non-cardiac vascular intervention and treatment requiring cardiac arrhythmias. The aim of the study was to include a total of 700 patients based on an expected event rate of 7-9%.

### Results

A total of 714 patients between 31 and 83 years of age were included in this study. As expected, the majority of patients was male (68.8%) presenting with stable angina pectoris symptoms (77%). Seventy-five patients were included with unstable angina symptoms and another 67 patients were included due to non ST-elevation myocardial infarction. Clinical decision making was left at the discretion of the treating cardiologist. Of the 714 included patients, 477 patients underwent PCI; 66 were referred for CABG; 89 patients were discharged with conservative treatment based on coronary angiography and an additional 83 patients were deferred based on non-significant (>0.80) FFR measurements of target lesions.

MicroRNA Profiling on 441 Patients

Data Analyses of 441 CD14⁺ Patients Samples Conformed Revealed Significant Associations of miRNA Expression with Clinical Phenotypes and Risk Factors

Data were normalized using median of medians by plate and hospital and expressed as average Ct value per miR. For several binary parameters, t-tests on Ct values were performed for each miR; for several categorical parameters, ANOVA was performed; for several continuous parameters, correlation analysis was performed. Logistic regression was performed on MACE controlling for smoking, gender, age and systolic blood pressure.

All miRs with significant associations with clinical data are depicted in Table 8.

TABLE 8
miRNAs with significant associations with clinical data.

| miRNA | Seq ID | Analyses sub groups | All 441 patients |
|---|---|---|---|
| hsa-miR-let-7d | 49 | vessel disease | |
| hsa-miR-let-7e | 50 | vessel disease, stable | MACE, number of diseased coronary vessels |
| | | CAD | |
| hsa-miR-let-7f | 2 | stable CAD | confirmed diagnosis CAD |
| hsa-miR-18b | 5 | CRP | syntax score males |
| hsa-miR-19a | 53 | vessel disease | |
| hsa-miR-19b | 54 | | Diabetes |
| hsa-miR-20a | 55 | Stable CAD | |
| hsa-miR-28-5p | 9 | Stable CAD | |
| hsa-miR-31 | 61 | | diabetes in females |
| hsa-miR-99a | 13 | | diabetes in females |
| hsa-miR-124 | 48 | stable CAD | |
| hsa-miR-125a- | 66 | diabetes | |
| 5p | | | |
| hsa-miR-126 | 67 | stable CAD | MACE, diabetes in females |
| hsa-miR-128 | 17 | stable CAD | MACE, confirmed diagnosis CAD |
| hsa-miR-132 | 68 | stablevs unstable CAD | |
| hsa-miR-133b | 21 | diabetes, familial | |
| | | hypercholesterolemia, | |
| | | stable CAD | |
| hsa-miR-145 | 25 | vessel disease | previous HF, number of diseased coronary |
| | | | vessels |
| hsa-miR-146-5p | 72 | CRP | previous CVA, TIA, PTCA, number of |
| | | | diseased coronary vessels |
| hsa-miR-146a | 71 | | previous MI, PTCA, number of diseased |
| | | | coronary vessels |
| hsa-miR-150 | 73 | stable CAD | diabetes in females |
| hsa-miR-155 | 84 | | syntax score males |
| hsa-miR-191 | 75 | vessel disease, stable | |
| | | CAD | |
| hsa-miR-193a- | 26 | vessel disease | |
| 5p | | | |
| hsa-miR-195 | 77 | vessel disease | syntax score females |
| hsa-miR-197 | 78 | vessel disease | |
| hsa-miR-221 | 80 | vessel disease | |
| hsa-miR-223 | 81 | vessel disease, stable | Diabetes, hypertension in females |
| | | CAD | |
| hsa-miR-342-3p | 29 | stablevs unstable CAD | |
| hsa-miR-365 | 30 | vessel disease, stable | syntax score males |
| | | CAD | |
| hsa-miR-376c | 47 | | number of diseased coronary vessels, |
| | | | confirmed diagnosis CAD |
| hsa-miR-422a | 31 | CRP, vessel disease | |
| hsa-miR-424 | 32 | CRP, stable CAD | |
| hsa-miR-486-3p | 88 | familial | confirmed diagnosis CAD |
| | | hypercholesterolemia | |
| hsa-miR-501-5p | 40 | vessel disease | |
| hsa-miR-574-3p | 42 | vessel disease | |
| hsa-miR-628-5p | 44 | vessel disease | |
| hsa-miR-671-3p | 89 | familial | |
| | | hypercholesterolemia | |
| hsa-miR-708 | 45 | | syntax score females |
| hsa-miR-885-5p | 46 | | number of diseased coronary vessels |
| hsa-miR-138 | 22 | CRP | |
| hsa-miR-15b | 4 | vessel disease | |
| hsa-miR-218 | 28 | CRP, stable CAD | |
| hsa-miR-449a | 33 | stable CAD, | |
| hsa-miR-130a | 18 | vessel disease | |

Legends of abbreviations used in the table:
CAD: Coronary artery disease.
MACE: Mirs associated with progression of Major Cardiovascular Event (Myocardioal infaction, need for re-PCI, cardiovascular death, cerebrovascular incident, need for coronary artery bypass graft.
CRP: C Reactive Protein
SYNTAX: The Syntax Score (SS) was introduced in the Synergy between Percutaneous Coronary Intervention with Taxus and Cardiac Surgery (SYNTAX) (see example 4)

### REFERENCE LIST

Allen J B, Wong H L, Guyre P M, Simon G L, Wahl S M. Association of circulating receptor Fc gamma RIII-positive monocytes in AIDS patients with elevated levels of transforming growth factor-beta. J Clin Invest. 1991; 87:1773-1779.
Auffray C, Fogg D, Garfa M, Elain G, Join-Lambert O, Kayal S, Sarnacki S, Cumano A, Lauvau G, Geissmann F. Monitoring of blood vessels and tissues by a population of monocytes with patrolling behavior. Science. 2007; 317:666-670.
Berezikov E, Thuemmler F, van Laake L W, Kondova I, Bontrop R, Cuppen E, Plasterk R H. Diversity of microRNAs in human and chimpanzee brain. Nature Genetics. 2006 December; 38(12):1375-7.
Braunersreuther V, Zernecke A, Arnaud C, Liehn E A, Steffens S, Shagdarsuren E, Bidzhekov K, Burger F, Pelli G, Luckow B, Mach F, Weber C. Ccr5 but not Ccr1 deficiency reduces development of diet-induced atherosclerosis in mice. Arterioscler Thromb Vasc Biol. 2007; 27:373-379.
Carthew R W, Sontheimer E J. Origins and Mechanisms of miRNAs and siRNAs. Cell. 2009; 136:642-655.
Fingerle G, Pforte A, Passlick B, Blumenstein M, Strobel M, Ziegler-Heitbrock H W. The novel subset of CD14+/CD16+ blood monocytes is expanded in sepsis patients. Blood. 1993; 82:3170-3176.
Hansson G K. Inflammation, atherosclerosis, and coronary artery disease. N Engl J Med. 2005; 352:1685-1695.
Havelange V, Garzon R. MicroRNAs: emerging key regulators of hematopoiesis. Am J Hematol. 2010; 85:935-942.
Nockher W A, Scherberich J E. Expanded CD14+CD16+ monocyte subpopulation in patients with acute and chronic infections undergoing hemodialysis. Infect Immun. 1998; 66:2782-2790.
O'Neill L A, Sheedy F J, McCoy C E. MicroRNAs: the fine-tuners of Toll-like receptor signalling. Nat Rev Immunol. 2011; 11:163-175.
Pearson T A, Mensah G A, Alexander R W, Anderson J L, Cannon R O 3rd, Criqui M, Fadl Y Y, Fortmann S P, Hong Y, Myers G L, Rifai N, Smith S C Jr, Taubert K, Tracy R P, Vinicor FMarkers of inflammation and cardiovascular disease: application to clinical and public health practice: A statement for healthcare professionals from the Centers for Disease Control and Prevention and the American Heart Association. Circulation (2003), 107: 499-511.
Rivier A, Pene J, Rabesandratana H, Chanez P, Bousquet J, Campbell A M. Blood monocytes of untreated asthmatics exhibit some features of tissue macrophages. Clin Exp Immunol. 1995; 100:314-318.
Serruys P W, Morice M C, Kappetein A P, Colombo A, Holmes D R, Mack M J, et al. Percutaneous coronary intervention versus coronary-artery bypass grafting for severe coronary artery disease. N Engl J Med 2009; 360:961-972.
Sianos G, Morel M A, Kappetein A P, Morice M C, Colombo A, Dawkins K, van den Brand M, Van Dyck N, Russell M E, Mohr F W, Serruys P W: The SYNTAX Score: an angiographic tool grading the complexity of coronary artery disease. Eurolntervention. 2005 August; 1(2):219-27.
Swirski F K, Libby P, Aikawa E, Alcaide P, Luscinskas F W, Weissleder R, Pittet M J. Ly-6Chi monocytes dominate hypercholesterolemia-associated monocytosis and give rise to macrophages in atheromata. J Clin Invest. 2007; 117:195-205.
Swirski F K, Pittet M J, Kircher M F, Aikawa E, Jaffer F A, Libby P, Weissleder R. Monocyte accumulation in mouse atherogenesis is progressive and proportional to extent of disease. Proc Natl Acad Sci USA. 2006; 103:10340-10345.
Ylitalo R, Oksala O, Yla-Herttuala S, Ylitalo P. Effects of clodronate (dichloromethylene bisphosphonate) on the development of experimental atherosclerosis in rabbits. J Lab Clin Med. 1994; 123:769-776.
Ziegler-Heitbrock L, Ancuta P, Crowe S, Dalod M, Grau V, Hart D N, Leenen P J, Liu Y J, MacPherson G, Randolph G J, Scherberich J, Schmitz J, Shortman K, Shortman K, Sozzani S, Strobl H, Zembala M, Austyn J M, Lutz M B. Blood. 2010; 21; 116(16):e74-80
Zhu H and Snyder M. Protein arrays and microarrays. Curr. Opin. Chem. Biol. 2001; 5: 40-45.

1. A diagnostic portfolio comprising or consisting of at least one or at least two or more nucleic acid molecules, their complements, equivalents and/or fragments thereof, said nucleic acid molecules, complements, equivalents and/or fragments thereof being:
at least one sequence comprising or consisting of sequences having at least 80% of sequence identity with sequences selected from the group consisting of: SEQ ID NO: 49, 50, 53, 25, 75, 26, 77, 78, 80, 81, 30, 31, 40, 42, 44, 4 and 18 (table 8),
at least two sequences comprising or consisting of sequences having at least 80% of sequence identity with sequences selected from the group consisting of: SEQ ID NO:1-92 (table 3),
at least one sequence comprising or consisting of sequences having at least 80% of sequence identity with sequences selected from the group consisting of: SEQ ID NO:29, 33, 43, 68, 71 and 90 (table 5, group a),
at least one sequence comprising or consisting of sequences having at least 80% of sequence identity with sequences selected from the group consisting of: SEQ ID NO: 27, 34, 37, 45, 47, and 79 (table 6, group b),
at least one sequence comprising or consisting of sequences having at least 80% of sequence identity with sequences selected from the group consisting of: SEQ ID NO: 3, 10, 14, 20, 46, 62, 66, 72,75,15, 17, 18, 19, 25, 26, 30, 39, 42, 44, 52, 53, 54, 55, 56, 63, 80, 81, 86, 88 and 91 (table 7, group c),
at least two sequences comprising or consisting of sequences having at least 80% of sequence identity with sequences selected from the group consisting of: SEQ ID NO:49-140 (table 4),
at least one sequence comprising or consisting of sequences having at least 80% of sequence identity with sequences selected from the group consisting of: SEQ ID NO: 50, 2, 55, 9, 48, 67, 17, 68, 21, 73, 75, 81, 29, 30, 32, 28 and 33 (table 8),
at least one sequence comprising or consisting of sequences having at least 80% of sequence identity with sequences selected from the group consisting of: SEQ ID NO: 5, 72, 31, 32, 22 and 28 (table 8),
at least one sequence comprising or consisting of sequences having at least 80% of sequence identity with sequences selected from the group consisting of: SEQ ID NO: 61, 13, 67, 73, 66, 54, 21 and 81 (table 8),
at least one sequence comprising or consisting of sequences having at least 80% of sequence identity with sequences selected from the group consisting of: SEQ ID NO: 61, 13, 67 and 73 (table 8), or
at least one sequence comprising or consisting of sequences having at least 80% of sequence identity with sequences selected from the group consisting of SEQ ID NO: 21, 88 and 89 (table 8), or
at least one sequence comprising or consisting of sequences having at least 80% of sequence identity with sequences selected from the group consisting of: SEQ ID NO: 47 and 46 (table 8).

2. A diagnostic portfolio according to claim 1 wherein,
a) group a) or table 5 further comprises a nucleic acid molecule, its complement, equivalent and/or fragment thereof, said nucleic acid molecule, complement, equivalent and/or fragment thereof comprising or consisting of a sequence having at least 80% of sequence identity with SEQ ID NO: 28,
c) group c) or table 7 further comprises at least one nucleic acid molecule, complement, equivalent and/or fragment thereof, said nucleic acid molecule, complement, equivalent and/or fragment thereof being selected from sequences having at least 80% of sequence identity with sequences selected from SEQ ID NO:13, 67, 73, 31, 24, 4, 65 and 84.

3. A diagnostic portfolio according to claim 1, in a matrix suitable for identifying the differential expression of a nucleic acid molecule contained therein.

4. A diagnostic portfolio according to claim 3, wherein said matrix is employed in a microarray and preferably wherein said microarray is an oligonucleotide microarray.

5. A kit for assessing the presence or absence of or the susceptibility to inflammation in an individual comprising reagents for detecting nucleic acid molecules, complements, and/or fragments thereof, said nucleic acid molecules being as identified in the diagnostic portfolio of claim 1, and optionally further comprising instructions.

6. A kit according to claim 5, further comprising reagents for conducting a microarray analysis and optionally further comprising a medium through which said nucleic acid molecules, their complements and/or fragments thereof are assayed, preferably wherein said medium is a microarray.

7. A method of assessing the presence or absence of or the susceptibility to inflammation in an individual using a diagnostic portfolio as defined in claim 1.

8. A method according to claim 7, comprising identifying differential modulation of a nucleic acid molecule (relative to the expression of a same nucleic acid molecule in a control).

9. A method of assessing whether an individual responds to a given treatment by a decrease or delay or absence of inflammation using a diagnostic portfolio as defined in claim 1.

10. A method according to claim 9, comprising identifying differential modulation of a nucleic acid molecule (relative to the expression of a same nucleic acid molecule in a control).

11. A method for identification of a substance capable of preventing, delaying, curing and/or treating a disease or a condition associated with inflammation in an individual, the method comprising the steps of:
(a) providing a test cell population capable of expressing a nucleic acid molecule as identified in claim 1,
(b) contacting the test cell population with the substance;
(c) determining the expression or activity level of a nucleic acid molecule as identified in step (a) in the test cell population contacted with the substance;
(d) comparing the expression or activity level determined in (c) with the expression or activity of corresponding nucleic acid molecule in a test cell population that is not contacted with the substance; and,
(e) identifying a substance that produces a difference in expression or activity level of a nucleic acid molecule as identified in step (a) between the test cell population that is contacted with the substance and the test cell population that is not contacted with the substance.

12. A diagnostic portfolio according to claim 2, in a matrix suitable for identifying the differential expression of a nucleic acid molecule contained therein.

13. A kit for assessing the presence or absence of or the susceptibility to inflammation in an individual comprising reagents for detecting nucleic acid molecules, complements, and/or fragments thereof, said nucleic acid molecules being as identified in the diagnostic portfolio of claim 4, and optionally further comprising instructions.

14. A kit according to claim 13, further comprising reagents for conducting a microarray analysis and optionally further comprising a medium through which said nucleic acid molecules, their complements and/or fragments thereof are assayed, preferably wherein said medium is a microarray.

15. A method of assessing the presence or absence of or the susceptibility to inflammation in an individual using a diagnostic portfolio as defined in claim 4.

16. A method of assessing the presence or absence of or the susceptibility to inflammation in an individual using a kit as defined in claim 5.

17. A method of assessing the presence or absence of or the susceptibility to inflammation in an individual using a kit as defined in claim 6.

18. A method according to claim 17, comprising identifying differential modulation of a nucleic acid molecule (relative to the expression of a same nucleic acid molecule in a control).

19. A method of assessing whether an individual responds to a given treatment by a decrease or delay or absence of inflammation using a diagnostic portfolio as defined in claim 4.

20. A method of assessing whether an individual responds to a given treatment by a decrease or delay or absence of inflammation using a diagnostic portfolio as defined in claim 5.

21. A method of assessing whether an individual responds to a given treatment by a decrease or delay or absence of inflammation using a diagnostic portfolio as defined in claim 6.

22. A method according to claim 21, comprising identifying differential modulation of a nucleic acid molecule (relative to the expression of a same nucleic acid molecule in a control).

23. A method for identification of a substance capable of preventing, delaying, curing and/or treating a disease or a condition associated with inflammation in an individual, the method comprising the steps of:
(a) providing a test cell population capable of expressing a nucleic acid molecule as identified in claim 2,
(b) contacting the test cell population with the substance;
(c) determining the expression or activity level of a nucleic acid molecule as identified in step (a) in the test cell population contacted with the substance;
(d) comparing the expression or activity level determined in (c) with the expression or activity of corresponding nucleic acid molecule in a test cell population that is not contacted with the substance; and,
(e) identifying a substance that produces a difference in expression or activity level of a nucleic acid molecule as identified in step (a) between the test cell population that is contacted with the substance and the test cell population that is not contacted with the substance.


SEQUENCE LISTING (140 sequences)

SEQ ID NO: 1 - RNA, 22 nt, Artificial
  hsa-miR-1 miRNA sequence
uggaauguaaagaaguauguau

SEQ ID NO: 2 - RNA, 22 nt, Artificial
  hsa-let-7f miRNA sequence
ugagguaguagauuguauaguu

SEQ ID NO: 3 - RNA, 23 nt, Artificial
  hsa-miR-10a miRNA sequence
uacccuguagauccgaauuugug

SEQ ID NO: 4 - RNA, 22 nt, Artificial
  hsa-miR-15b miRNA sequence
uagcagcacaucaugguuuaca

SEQ ID NO: 5 - RNA, 23 nt, Artificial
  hsa-miR-18b miRNA sequence
uaaggugcaucuagugcaguuag

SEQ ID NO: 6 - RNA, 22 nt, Artificial
  hsa-miR-26a miRNA sequence
uucaaguaauccaggauaggcu

SEQ ID NO: 7 - RNA, 21 nt, Artificial
  hsa-miR-27b miRNA sequence
uucacaguggcuaaguucugc

SEQ ID NO: 8 - RNA, 22 nt, Artificial
  hsa-miR-28-3p miRNA sequence
cacuagauugugagcuccugga

SEQ ID NO: 9 - RNA, 22 nt, Artificial
  hsa-miR-28-5p miRNA sequence
aaggagcucacagucuauugag

SEQ ID NO: 10 - RNA, 22 nt, Artificial
  hsa-miR-32 miRNA sequence
uauugcacauuacuaaguugca

SEQ ID NO: 11 - RNA, 22 nt, Artificial
  hsa-miR-92a miRNA sequence
uauugcacuugucccggccugu

SEQ ID NO: 12 - RNA, 22 nt, Artificial
  hsa-miR-98 miRNA sequence
ugagguaguaaguuguauuguu

SEQ ID NO: 13 - RNA, 22 nt, Artificial
  hsa-miR-99a miRNA sequence
aacccguagauccgaucuugug

SEQ ID NO: 14 - RNA, 22 nt, Artificial
  hsa-miR-99b miRNA sequence
cacccguagaaccgaccuugcg

SEQ ID NO: 15 - RNA, 22 nt, Artificial
  hsa-miR-100 miRNA sequence
aacccguagauccgaacuugug

SEQ ID NO: 16 - RNA, 22 nt, Artificial
  hsa-miR-125b miRNA sequence
ucccugagacccuaacuuguga

SEQ ID NO: 17 - RNA, 21 nt, Artificial
  hsa-miR-128 miRNA sequence
ucacagugaaccggucucuuu

SEQ ID NO: 18 - RNA, 22 nt, Artificial
  hsa-miR-130a miRNA sequence
cagugcaauguuaaaagggcau

SEQ ID NO: 19 - RNA, 22 nt, Artificial
  hsa-miR-130b miRNA sequence
cagugcaaugaugaaagggcau

SEQ ID NO: 20 - RNA, 22 nt, Artificial
  hsa-miR-133a miRNA sequence
uuugguccccuucaaccagcug

SEQ ID NO: 21 - RNA, 22 nt, Artificial
  hsa-miR-133b miRNA sequence
uuugguccccuucaaccagcua

SEQ ID NO: 22 - RNA, 23 nt, Artificial
  hsa-miR-138 miRNA sequence
agcugguguugugaaucaggccg

SEQ ID NO: 23 - RNA, 22 nt, Artificial
  hsa-miR-140-5p miRNA sequence
cagugguuuuacccuaugguag

SEQ ID NO: 24 - RNA, 21 nt, Artificial
  hsa-miR-142-5p miRNA sequence
cauaaaguagaaagcacuacu

SEQ ID NO: 25 - RNA, 23 nt, Artificial
  hsa-miR-145 miRNA sequence
guccaguuuucccaggaaucccu

SEQ ID NO: 26 - RNA, 22 nt, Artificial
  hsa-miR-193a-5p miRNA sequence
ugggucuuugcgggcgagauga

SEQ ID NO: 27 - RNA, 22 nt, Artificial
  hsa-miR-200a miRNA sequence
uaacacugucugguaacgaugu

SEQ ID NO: 28 - RNA, 21 nt, Artificial
  hsa-miR-218 miRNA sequence
uugugcuugaucuaaccaugu

SEQ ID NO: 29 - RNA, 23 nt, Artificial
  hsa-miR-342-3p miRNA sequence
ucucacacagaaaucgcacccgu

SEQ ID NO: 30 - RNA, 22 nt, Artificial
  hsa-miR-365 miRNA sequence
uaaugccccuaaaaauccuuau

SEQ ID NO: 31 - RNA, 22 nt, Artificial
  hsa-miR-422a miRNA sequence
acuggacuuagggucagaaggc

SEQ ID NO: 32 - RNA, 22 nt, Artificial
  hsa-miR-424 miRNA sequence
cagcagcaauucauguuuugaa

SEQ ID NO: 33 - RNA, 22 nt, Artificial
  hsa-miR-449a miRNa sequence
uggcaguguauuguuagcuggu

SEQ ID NO: 34 - RNA, 22 nt, Artificial
  hsa-miR-449b miRNA sequence
aggcaguguauuguuagcuggc

SEQ ID NO: 35 - RNA, 22 nt, Artificial
  hsa-miR-450b-5p miRNA sequence
uuuugcaauauguuccugaaua

SEQ ID NO: 36 - RNA, 22 nt, Artificial
  hsa-miR-451 miRNA sequence
aaaccguuaccauuacugaguu

SEQ ID NO: 37 - RNA, 22 nt, Artificial
  hsa-miR-487b miRNA sequence
aaucguacagggucauccacuu

SEQ ID NO: 38 - RNA, 22 nt, Artificial
  hsa-miR-491-5p miRNA sequence
aguggggaacccuuccaugagg

SEQ ID NO: 39 - RNA, 23 nt, Artificial
  hsa-miR-500 miRNA sequence
uaauccuugcuaccugggugaga

SEQ ID NO: 40 - RNA, 22 nt, Artificial
  hsa-miR-501-5p miRNA sequence
aauccuuugucccugggugaga

SEQ ID NO: 41 - RNA, 23 nt, Artificial
  hsa-miR-503 miRNA sequence
uagcagcgggaacaguucugcag

SEQ ID NO: 42 - RNA, 22 nt, Artificial
  hsa-miR-574-3p miRNA sequence
cacgcucaugcacacacccaca

SEQ ID NO: 43 - RNA, 22 nt, Artificial
  hsa-miR-590-5p miRNA sequence
gagcuuauucauaaaagugcag

SEQ ID NO: 44 - RNA, 22 nt, Artificial
  hsa-miR-628-5p miRNA sequence
augcugacauauuuacuagagg

SEQ ID NO: 45 - RNA, 23 nt, Artificial
  hsa-miR-708 miRNA sequence
aaggagcuuacaaucuagcuggg

SEQ ID NO: 46 - RNA, 22 nt, Artificial
  hsa-miR-885-5p miRNA sequence
uccauuacacuacccugccucu

SEQ ID NO: 47 - RNA, 21 nt, Artificial
  hsa-miR-376c miRNA sequence
aacauagaggaaauuccacgu

SEQ ID NO: 48 - RNA, 20 nt, Artificial
  hsa-mir-124  miRNA sequence
uaaggcacgcggugaaugcc

SEQ ID NO: 49 - RNA, 22 nt, Artificial
  hsa-let-7d miRNA sequence
agagguaguagguugcauaguu

SEQ ID NO: 50 - RNA, 22 nt, Artificial
  hsa-let-7e miRNA sequence
ugagguaggagguuguauaguu

SEQ ID NO: 51 - RNA, 22 nt, Artificial
  hsa-let-7g miRNA sequence
ugagguaguaguuuguacaguu

SEQ ID NO: 52 - RNA, 23 nt, Artificial
  hsa-miR-17 miRNA sequence
caaagugcuuacagugcagguag

SEQ ID NO: 53 - RNA, 23 nt, Artificial
  hsa-miR-19a miRNA sequence
ugugcaaaucuaugcaaaacuga

SEQ ID NO: 54 - RNA, 23 nt, Artificial
  hsa-miR-19b miRNA sequence
ugugcaaauccaugcaaaacuga

SEQ ID NO: 55 - RNA, 23 nt, Artificial
  hsa-miR-20a miRNA sequence
uaaagugcuuauagugcagguag

SEQ ID NO: 56 - RNA, 23 nt, Artificial
  hsa-miR-20b miRNA sequence
caaagugcucauagugcagguag

SEQ ID NO: 57 - RNA, 22 nt, Artificial
  hsa-miR-21 miRNA sequence
uagcuuaucagacugauguuga

SEQ ID NO: 58 - RNA, 21 nt, Artificial
  hsa-miR-26b miRNA sequence
uucaaguaauucaggauaggu

SEQ ID NO: 59 - RNA, 21 nt, Artificial
  hsa-miR-27a miRNA sequence
uucacaguggcuaaguuccgc

SEQ ID NO: 60 - RNA, 22 nt, Artificial
  hsa-miR-29a miRNA sequence
uagcaccaucugaaaucgguua

SEQ ID NO: 61 - RNA, 21 nt, Artificial
  hsa-miR-31 miRNA sequence
aggcaagaugcuggcauagcu

SEQ ID NO: 62 - RNA, 22 nt, Artificial
  hsa-miR-34a miRNA sequence
uggcagugucuuagcugguugu

SEQ ID NO: 63 - RNA, 23 nt, Artificial
  hsa-miR-93 miRNA sequence
caaagugcuguucgugcagguag

SEQ ID NO: 64 - RNA, 23 nt, Artificial
  hsa-miR-103 miRNA sequence
agcagcauuguacagggcuauga

SEQ ID NO: 65 - RNA, 21 nt, Artificial
  hsa-miR-106b miRNA sequence
uaaagugcugacagugcagau

SEQ ID NO: 66 - RNA, 23 nt, Artificial
  hsa-miR-125a-5p miRNA sequence
ucccugagacccuuuaaccugug

SEQ ID NO: 67 - RNA, 22 nt, Artificial
  hsa-miR-126 miRNA sequence
ucguaccgugaguaauaaugcg

SEQ ID NO: 68 - RNA, 22 nt, Artificial
  hsa-miR-132 miRNA sequence
uaacagucuacagccauggucg

SEQ ID NO: 69 - RNA, 22 nt, Artificial
  hsa-miR-139-5p miRNA sequence
ucuacagugcacgugucuccag

SEQ ID NO: 70 - RNA, 23 nt, Artificial
  hsa-miR-142-3p miRNA sequence
uguaguguuuccuacuuuaugga

SEQ ID NO: 71 - RNA, 22 nt, Artificial
  hsa-miR-146a miRNA sequence
ugagaacugaauuccauggguu

SEQ ID NO: 72 - RNA, 22 nt, Artificial
  hsa-miR-146b-5p miRNA sequence
ugagaacugaauuccauaggcu

SEQ ID NO: 73 - RNA, 22 nt, Artificial
  hsa-miR-150 miRNA sequence
ucucccaacccuuguaccagug

SEQ ID NO: 74 - RNA, 23 nt, Artificial
  hsa-miR-181a miRNA sequence
aacauucaacgcugucggugagu

SEQ ID NO: 75 - RNA, 23 nt, Artificial
  hsa-miR-191 miRNA sequence
caacggaaucccaaaagcagcug

SEQ ID NO: 76 - RNA, 22 nt, Artificial
  hsa-miR-193b miRNA sequence
aacuggcccucaaagucccgcu

SEQ ID NO: 77 - RNA, 21 nt, Artificial
  hsa-miR-195 miRNA sequence
uagcagcacagaaauauuggc

SEQ ID NO: 78 - RNA, 22 nt, Artificial
  hsa-miR-197 miRNA sequence
uucaccaccuucuccacccagc

SEQ ID NO: 79 - RNA, 22 nt, Artificial
  hsa-miR-210 miRNA sequence
cugugcgugugacagcggcuga

SEQ ID NO: 80 - RNA, 23 nt, Artificial
  hsa-miR-221 miRNA sequence
agcuacauugucugcuggguuuc

SEQ ID NO: 81 - RNA, 22 nt, Artificial
  hsa-miR-223 miRNA sequence
ugucaguuugucaaauacccca

SEQ ID NO: 82 - RNA, 22 nt, Artificial
  hsa-miR-328 miRNA sequence
cuggcccucucugcccuuccgu

SEQ ID NO: 83 - RNA, 21 nt, Artificial
  hsa-miR-331-3p miRNA sequence
gccccugggccuauccuagaa

SEQ ID NO: 84 - RNA, 23 nt, Artificial
  hsa-miR-155 miRNA sequence
uuaaugcuaaucgugauaggggu

SEQ ID NO: 85 - RNA, 22 nt, Artificial
  hsa-let-7b miRNA sequence
ugagguaguagguugugugguu

SEQ ID NO: 86 - RNA, 22 nt, Artificial
  hsa-miR-374b miRNA sequence
auauaauacaaccugcuaagug

SEQ ID NO: 87 - RNA, 23 nt, Artificial
  hsa-miR-423-5p miRNA sequence
ugaggggcagagagcgagacuuu

SEQ ID NO: 88 - RNA, 21 nt, Artificial
  hsa-miR-486-3p miRNA sequence
cggggcagcucaguacaggau

SEQ ID NO: 89 - RNA, 21 nt, Artificial
  hsa-miR-671-3p miRNA sequence
uccgguucucagggcuccacc

SEQ ID NO: 90 - RNA, 21 nt, Artificial
  hsa-miR-212 miRNA sequence
uaacagucuccagucacggcc

SEQ ID NO: 91 - RNA, 22 nt, Artificial
  hsa-miR-345 miRNA sequence
gcugacuccuaguccagggcuc

SEQ ID NO: 92 - RNA, 21 nt, Artificial
  hsa-let-7i miRNA sequence
ugagguaguaguuugugcugu

SEQ ID NO: 93 - RNA, 22 nt, Artificial
  hsa-let-7a miRNA sequence
ugagguaguagguuguauaguu

SEQ ID NO: 94 - RNA, 22 nt, Artificial
  hsa-miR-15a miRNA sequence
uagcagcacauaaugguuugug

SEQ ID NO: 95 - RNA, 23 nt, Artificial
  hsa-miR-18a miRNA sequence
uaaggugcaucuagugcagauag

SEQ ID NO: 96 - RNA, 22 nt, Artificial
  hsa-miR-24 miRNA sequence
uggcucaguucagcaggaacag

SEQ ID NO: 97 - RNA, 22 nt, Artificial
  hsa-miR-29c miRNA sequence
uagcaccauuugaaaucgguua

SEQ ID NO: 98 - RNA, 22 nt, Artificial
  hsa-miR-30b miRNA sequence
uguaaacauccuacacucagcu

SEQ ID NO: 99 - RNA, 23 nt, Artificial
  hsa-miR-34c-5p miRNA sequence
aggcaguguaguuagcugauugc

SEQ ID NO: 100 - RNA, 23 nt, Artificial
  hsa-miR-106a miRNA sequence
aaaagugcuuacagugcagguag

SEQ ID NO: 101 - RNA, 23 nt, Artificial
  hsa-miR-135b miRNA sequence
uauggcuuuucauuccuauguga

SEQ ID NO: 102 - RNA, 22 nt, Artificial
  hsa-miR-185 miRNA sequence
uggagagaaaggcaguuccuga

SEQ ID NO: 103 - RNA, 22 nt, Artificial
  hsa-miR-186 miRNA sequence
caaagaauucuccuuuugggcu

SEQ ID NO: 104 - RNA, 22 nt, Artificial
  hsa-miR-199a-3p miRNA sequence
acaguagucugcacauugguua

SEQ ID NO: 105 - RNA, 23 nt, Artificial
  hsa-miR-199b-5p miRNA sequence
cccaguguuuagacuaucuguuc

SEQ ID NO: 106 - RNA, 23 nt, Artificial
  hsa-miR-200c miRNA sequence
uaauacugccggguaaugaugga

SEQ ID NO: 107 - RNA, 21 nt, Artificial
  hsa-miR-222 miRNA sequence
agcuacaucuggcuacugggu

SEQ ID NO: 108 - RNA, 20 nt, Artificial
  hsa-miR-326 miRNA sequence
ccucugggcccuuccuccag

SEQ ID NO: 109 - RNA, 23 nt, Artificial
  hsa-miR-330-3p miRNA sequence
gcaaagcacacggccugcagaga

SEQ ID NO: 110 - RNA, 23 nt, Artificial
  hsa-miR-335 miRNA sequence
ucaagagcaauaacgaaaaaugu

SEQ ID NO: 111 - RNA, 21 nt, Artificial
  hsa-miR-337-5p miRNA sequence
gaacggcuucauacaggaguu

SEQ ID NO: 112 - RNA, 23 nt, Artificial
  hsa-miR-339-5p miRNA sequence
ucccuguccuccaggagcucacg

SEQ ID NO: 113 - RNA, 22 nt, Artificial
  hsa-miR-340 miRNA sequence
uuauaaagcaaugagacugauu

SEQ ID NO: 114 - RNA, 22 nt, Artificial
  hsa-miR-374a miRNA sequence
uuauaauacaaccugauaagug

SEQ ID NO: 115 - RNA, 23 nt, Artificial
  hsa-miR-409-5p miRNA sequence
agguuacccgagcaacuuugcau

SEQ ID NO: 116 - RNA, 23 nt, Artificial
  hsa-miR-425 miRNA sequence
aaugacacgaucacucccguuga

SEQ ID NO: 117 - RNA, 23 nt, Artificial
  hsa-miR-454 miRNA sequence
uagugcaauauugcuuauagggu

SEQ ID NO: 118 - RNA, 22 nt, Artificial
  hsa-miR-455-5p miRNA sequence
uaugugccuuuggacuacaucg

SEQ ID NO: 119 - RNA, 22 nt, Artificial
  hsa-miR-484 miRNA sequence
ucaggcucaguccccucccgau

SEQ ID NO: 120 - RNA, 22 nt, Artificial
  hsa-miR-532-5p miRNA sequence
caugccuugaguguaggaccgu

SEQ ID NO: 121 - RNA, 23 nt, Artificial
  hsa-miR-542-5p miRNA sequence
ucggggaucaucaugucacgaga

SEQ ID NO: 122 - RNA, 23 nt, Artificial
  hsa-miR-582-5p miRNA sequence
uuacaguuguucaaccaguuacu

SEQ ID NO: 123 - RNA, 22 nt, Artificial
  hsa-miR-598 miRNA sequence
uacgucaucguugucaucguca

SEQ ID NO: 124 - RNA, 21 nt, Artificial
  hsa-miR-625 miRNA sequence
agggggaaaguucuauagucc

SEQ ID NO: 125 - RNA, 22 nt, Artificial
  hsa-miR-654-5p miRNA sequence
uggugggccgcagaacaugugc

SEQ ID NO: 126 - RNA, 22 nt, Artificial
  hsa-miR-660 miRNA sequence
uacccauugcauaucggaguug

SEQ ID NO: 127 - RNA, 22 nt, Artificial
  hsa-miR-744 miRNA sequence
ugcggggcuagggcuaacagca

SEQ ID NO: 128 - RNA, 23 nt, Artificial
  hsa-miR-886-5p miRNA sequence
cgggucggaguuagcucaagcgg

SEQ ID NO: 129 - RNA, 21 nt, Artificial
  hsa-mir-1308 miRNA sequence
gcaugggugguucagugguag

SEQ ID NO: 130 - RNA, 23 nt, Artificial
  hsa-mir-181b miRNA sequence
aacauucauugcugucggugggu

SEQ ID NO: 131 - RNA, 24 nt, Artificial
  hsa-mir-1979 miRNA sequence
cucccacugcuucacuugacuagc

SEQ ID NO: 132 - RNA, 23 nt, Artificial
  hsa-mir-2110 miRNA sequence
uuggggaaacggccgcugaguga

SEQ ID NO: 133 - RNA, 24 nt, Artificial
  hsa-mir-320a miRNA sequence
aaaagcuggguugagagggcgaaa

SEQ ID NO: 134 - RNA, 20 nt, Artificial
  hsa-mir-378c miRNA sequence
acuggacuuggagucagaag

SEQ ID NO: 135 - RNA, 23 nt, Artificial
  hsa-mir-4284 miRNA sequence
gcucacaucaccccauaaacaaa

SEQ ID NO: 136 - RNA, 20 nt, Artificial
  hsa-mir-196a miRNA sequence
uagguaguuucauguuguug

SEQ ID NO: 137 - RNA, 22 nt, Artificial
  hsa-mir-1249 miRNA sequence
acgcccuucccccccuucuuca

SEQ ID NO: 138 - RNA, 22 nt, Artificial
  hsa-mir-1271 miRNA sequence
cuuggcaccuagcaagcacuca

SEQ ID NO: 139 - RNA, 22 nt, Artificial
  hsa-mir-378 miRNA sequence
cuccugacuccagguccugugu

SEQ ID NO: 140 - RNA, 22 nt, Artificial
  hsa-mir-942 miRNA sequence
ucuucucuguuuuggccaugug